SEQUENCE LISTING (42 sequences)

SEQ ID NO: 1 - protein, 5 aa, Homo sapiens
SKECF

SEQ ID NO: 2 - protein, 311 aa, Homo sapiens
AKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 3 - protein, 10 aa, synthetic construct
  REGION 1..10: note = CDR1 of the variable domain of the heavy chain of the anti-sST2 antibody
GGSFSGYYWS

SEQ ID NO: 4 - protein, 16 aa, synthetic construct
  REGION 1..16: note = CDR2 of the variable domain of the heavy chain of the antibody anti-sST2
EINHSGSTNYNPSLKS

SEQ ID NO: 5 - protein, 11 aa, synthetic construct
  REGION 1..11: note = CDR3of the variable domain of the heavy chain of the antibody anti-sST2
ARGRGLVRFDS

SEQ ID NO: 6 - protein, 12 aa, synthetic construct
  REGION 1..12: note = CDR1 of the variable domain of the light chain of the antibody anti-sST2
RASQFVGSSSVA

SEQ ID NO: 7 - protein, 7 aa, synthetic construct
  REGION 1..7: note = CDR2 of the variable domain of the light chain of the antibody anti-sST2
AAVSRAS

SEQ ID NO: 8 - protein, 9 aa, synthetic construct
  REGION 1..9: note = CDR3 of the variable domain of the light chain of the antibody anti-sST2
LQYGSALYT

SEQ ID NO: 9 - protein, 116 aa, synthetic construct
  REGION 1..116: note = Variable domain of the heavy chain of the antibody anti-sST2
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRGLVRFDSWGQGTTVTV

SEQ ID NO: 10 - protein, 108 aa, synthetic construct
  REGION 1..108: note = Variable domain of the light chain of the antibody anti-sST2
EIVLTQSPGTLSLSPGDRATLSCRASQFVGSSSVAWYQQRPGQAPRLLIYAAVSRASGIPDRFSGGGSGTDFTLTISRLEPEDSAVYYCLQYGSALYTFGQGTKLEIK

SEQ ID NO: 11 - protein, 241 aa, synthetic construct
  REGION 1..241: note = Single-chain variable fragment (scFv) anti-sST2
  DOMAIN 1..116: note = Variable domain of the heavy chain
  SITE 117..133: note = Linker
  DOMAIN 134..241: note = Variable domain of the light chain
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRGLVRFDSWGQGTTVTVSSGGGGSGGGGSGGGGSEIVLTQSPGTLSLSPGDRATLSCRASQFVGSSSVAWYQQRPGQAPRLLIYAAVSRASGIPDRFSGGGSGTDFTLTISRLEPEDSAVYYCLQYGSALYTFGQGTKLEIK

SEQ ID NO: 12 - protein, 241 aa, synthetic construct
  REGION 1..241: note = Single-chain variable fragment (scFv) anti-sST2, variant E51Q
  DOMAIN 1..116: note = Variable domain of the heavy chain
  SITE 117..133: note = Linker
  DOMAIN 134..241: note = Variable domain of the light chain
MQVQLQQWGAGLLKPSETLSLTCAVYGGSFSGYYWSWIRQPPGKGLEWIGQINHSGSTNYNPSLKSRVTVSLDTSKNQFSLKLSSVTAADTAVYYCARGRGLVRFDSWGQGTTVTVSSGGGGSGGGGSGGGGSEIVLTQSPGTLSLSPGDRATLSCRASQFVGSSSVAWYQQRPGQAPRLLIYAAVSRASGIPDRFSGGGSGTDFTLTISRLEPEDSAVYYCLQYGSALYTFGQGTKLEIK

SEQ ID NO: 13 - protein, 241 aa, synthetic construct
  REGION 1..241: note = Single-chain variable fragment (scFv) anti-sST2, variant G101W
  DOMAIN 1..116: note = Variable domain of the heavy chain
  SITE 117..133: note = Linker
  DOMAIN 134..241: note = Variable domain of the light chain
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRWLVRFDSWGQGTTVTVSSGGGGSGGGGSGGGGSEIVLTQSPGTLSLSPGDRATLSCRASQFVGSSSVAWYQQRPGQAPRLLIYAAVSRASGIPDRFSGGGSGTDFTLTISRLEPEDSAVYYCLQYGSALYTFGQGTKLEIK

SEQ ID NO: 14 - protein, 241 aa, synthetic construct
  REGION 1..241: note = Single-chain variable fragment (scFv) anti-sST2, variant A228H/S166R
  DOMAIN 1..116: note = Variable domain of the heavy chain
  SITE 117..133: note = Linker
  DOMAIN 134..241: note = Variable domain of the light chain
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRGLVRFDSWGQGTTVTVSSGGGGSGGGGSGGGGSEIVLTQSPGTLSLSPGDRATLSCRASQFVGSSRVAWYQQRPGQAPRLLIYAAVSRASGIPDRFSGGGSGTDFTLTISRLEPEDSAVYYCLQYGSHLYTFGQGTKLEIK

SEQ ID NO: 15 - protein, 241 aa, synthetic construct
  REGION 1..241: note = Single-chain variable fragment (scFv) anti-sST2, variant A184Y
  DOMAIN 1..116: note = Variable domain of the heavy chain
  SITE 117..133: note = Linker
  DOMAIN 134..241: note = Variable domain of the light chain
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRGLVRFDSWGQGTTVTVSSGGGGSGGGGSGGGGSEIVLTQSPGTLSLSPGDRATLSCRASQFVGSSSVAWYQQRPGQAPRLLIYYAVSRASGIPDRFSGGGSGTDFTLTISRLEPEDSAVYYCLQYGSALYTFGQGTKLEIK

SEQ ID NO: 16 - DNA, 30 bp, synthetic construct
  misc_feature 1..30: note = Nucleotide sequence of the CDR1 of the variable domain of the heavy chain of the anti-sST2 antibody
  source 1..30: mol_type = other DNA
ggcggcagcttttctggctactattggagc

SEQ ID NO: 17 - DNA, 48 bp, synthetic construct
  misc_feature 1..48: note = Nucleotide sequence of the CDR2 of the variable domain of the heavy chain of the anti-sST2 antibody
  source 1..48: mol_type = other DNA
gagatcaaccactccggcagcaccaactataatccttccctgaagagc

SEQ ID NO: 18 - DNA, 33 bp, synthetic construct
  misc_feature 1..33: note = Nucleotide sequence of the CDR3 of the variable domain of the heavy chain of the anti-sST2 antibody
  source 1..33: mol_type = other DNA
gctaggggaaggggactggtgaggttcgattct

SEQ ID NO: 19 - DNA, 36 bp, synthetic construct
  misc_feature 1..36: note = Nucleotide sequence of the CDR1 of the variable domain of the light chain of the antibody anti-sST2
  source 1..36: mol_type = other DNA
cgggcttctcagtttgtgggctccagctctgtggcc

SEQ ID NO: 20 - DNA, 21 bp, synthetic construct
  misc_feature 1..21: note = Nucleotide sequence of the CDR2 of the variable domain of the light chain of the antibody anti-sST2
  source 1..21: mol_type = other DNA
gccgccgtgtccagggcctcc

SEQ ID NO: 21 - DNA, 27 bp, synthetic construct
  misc_feature 1..27: note = Nucleotide sequence of the CDR3 of the variable domain of the light chain of the antibody anti-sST2
  source 1..27: mol_type = other DNA
ctgcagtacggctccgctctgtatacc

SEQ ID NO: 22 - DNA, 348 bp, synthetic construct
  misc_feature 1..348: note = Nucleotide sequence of the variable domain of the heavy chain of the anti-sST2 antibody
  source 1..348: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggcgaaattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgtggtctggtgcgttttgatagctggggccagggtaccaccgttaccgtt

SEQ ID NO: 23 - DNA, 348 bp, synthetic construct
  misc_feature 1..348: note = Nucleotide sequence of the variable domain of the heavy chain of anti-sST2 antibody, variant E51Q
  source 1..348: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggccagattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgtggtctggtgcgttttgatagctggggccagggtaccaccgttaccgtt

SEQ ID NO: 24 - DNA, 348 bp, synthetic construct
  misc_feature 1..348: note = Nucleotide sequence of the variable domain of the heavy chain of the anti-sST2 antibody, variant G101W
  source 1..348: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggcgaaattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgttggctggtgcgttttgatagctggggccagggtaccaccgttaccgtt

SEQ ID NO: 25 - DNA, 324 bp, synthetic construct
  misc_feature 1..324: note = Nucleotide sequence of the variable domain of the light chain of the anti-sST2 antibody
  source 1..324: mol_type = other DNA
gagattgttctgacccaaagcccgggtaccctgagcctgagcccgggtgatcgtgcgaccctgagctgccgtgcgagccagttcgtgggtagcagcagcgtggcgtggtaccagcaacgtccgggtcaagcgccgcgtctgctgatctatgcggctgtgagccgtgcgagcggtattccggaccgtttcagcggtggcggtagcggtaccgattttaccctgaccatcagccgtctggagccggaagatagcgcggtgtactattgcctgcagtacggtagcgcgctgtatacctttggccaaggtaccaagctggaaattaaa

SEQ ID NO: 26 - DNA, 324 bp, synthetic construct
  misc_feature 1..324: note = Nucleotide sequence of the variable domain of the light chain of the anti-sST2 antibody, variant A228H/S166R
  source 1..324: mol_type = other DNA
gagattgttctgacccaaagcccgggtaccctgagcctgagcccgggtgatcgtgcgaccctgagctgccgtgcgagccagttcgtgggtagcagccgtgtggcgtggtaccagcaacgtccgggtcaagcgccgcgtctgctgatctatgcggctgtgagccgtgcgagcggtattccggaccgtttcagcggtggcggtagcggtaccgattttaccctgaccatcagccgtctggagccggaagatagcgcggtgtactattgcctgcagtacggtagccatctgtatacctttggccaaggtaccaagctggaaattaaa

SEQ ID NO: 27 - DNA, 324 bp, synthetic construct
  misc_feature 1..324: note = Nucleotide sequence of the variable domain of the light chain of the anti-sST2 antibody, variant A184Y
  source 1..324: mol_type = other DNA
gagattgttctgacccaaagcccgggtaccctgagcctgagcccgggtgatcgtgcgaccctgagctgccgtgcgagccagttcgtgggtagcagcagcgtggcgtggtaccagcaacgtccgggtcaagcgccgcgtctgctgatctattatgctgtgagccgtgcgagcggtattccggaccgtttcagcggtggcggtagcggtaccgattttaccctgaccatcagccgtctggagccggaagatagcgcggtgtactattgcctgcagtacggtagcgcgctgtatacctttggccaaggtaccaagctggaaattaaa

SEQ ID NO: 28 - DNA, 723 bp, synthetic construct
  misc_feature 1..723: note = Nucleotide sequence of the single-chain variable fragment (scFv) anti-sST2
  V_region 1..348: note = Variable region of the heavy chain
  misc_feature 349..399: note = Linker
  V_region 400..723: note = Variable region of the light chain
  source 1..723: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggcgaaattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgtggtctggtgcgttttgatagctggggccagggtaccaccgttaccgttagcagcggtggcggtggcagcggtggcggtggcagcggtggcggtggcagcgagattgttctgacccaaagcccgggtaccctgagcctgagcccgggtgatcgtgcgaccctgagctgccgtgcgagccagttcgtgggtagcagcagcgtggcgtggtaccagcaacgtccgggtcaagcgccgcgtctgctgatctatgcggctgtgagccgtgcgagcggtattccggaccgtttcagcggtggcggtagcggtaccgattttaccctgaccatcagccgtctggagccggaagatagcgcggtgtactattgcctgcagtacggtagcgcgctgtatacctttggccaaggtaccaagctggaaattaaa

SEQ ID NO: 29 - DNA, 723 bp, synthetic construct
  misc_feature 1..723: note = Nucleotide sequence of the single-chain variable fragment (scFv) anti-sST2, variant E51Q
  V_region 1..348: note = Regin variable de la cadena pesada (Var E50Q)
  misc_feature 349..399: note = Enlazador
  V_region 400..723: note = Regin variable de la cadena ligera (Var E50Q)
  source 1..723: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggccagattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgtggtctggtgcgttttgatagctggggccagggtaccaccgttaccgttagcagcggtggcggtggcagcggtggcggtggcagcggtggcggtggcagcgagattgttctgacccaaagcccgggtaccctgagcctgagcccgggtgatcgtgcgaccctgagctgccgtgcgagccagttcgtgggtagcagcagcgtggcgtggtaccagcaacgtccgggtcaagcgccgcgtctgctgatctatgcggctgtgagccgtgcgagcggtattccggaccgtttcagcggtggcggtagcggtaccgattttaccctgaccatcagccgtctggagccggaagatagcgcggtgtactattgcctgcagtacggtagcgcgctgtatacctttggccaaggtaccaagctggaaattaaa

SEQ ID NO: 30 - DNA, 723 bp, synthetic construct
  misc_feature 1..723: note = Nucleotide sequence of the single-chain variable fragment (scFv) anti-sST2, variant G101W
  V_region 1..348: note = Variable region of the heavy chain
  misc_feature 349..399: note = Linker
  V_region 400..723: note = Variable region of the light chain
  source 1..723: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggcgaaattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgttggctggtgcgttttgatagctggggccagggtaccaccgttaccgttagcagcggtggcggtggcagcggtggcggtggcagcggtggcggtggcagcgagattgttctgacccaaagcccgggtaccctgagcctgagcccgggtgatcgtgcgaccctgagctgccgtgcgagccagttcgtgggtagcagcagcgtggcgtggtaccagcaacgtccgggtcaagcgccgcgtctgctgatctatgcggctgtgagccgtgcgagcggtattccggaccgtttcagcggtggcggtagcggtaccgattttaccctgaccatcagccgtctggagccggaagatagcgcggtgtactattgcctgcagtacggtagcgcgctgtatacctttggccaaggtaccaagctggaaattaaa

SEQ ID NO: 31 - DNA, 723 bp, synthetic construct
  misc_feature 1..723: note = Nucleotide sequence of the single-chain variable fragment (scFv) anti-sST2, variant A228H/S166R
  V_region 1..348: note = Variable region of the heavy chain
  misc_feature 349..399: note = Linker
  V_region 400..723: note = Variable region of the light chain
  source 1..723: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggcgaaattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgtggtctggtgcgttttgatagctggggccagggtaccaccgttaccgttagcagcggtggcggtggcagcggtggcggtggcagcggtggcggtggcagcgagattgttctgacccaaagcccgggtaccctgagcctgagcccgggtgatcgtgcgaccctgagctgccgtgcgagccagttcgtgggtagcagccgtgtggcgtggtaccagcaacgtccgggtcaagcgccgcgtctgctgatctatgcggctgtgagccgtgcgagcggtattccggaccgtttcagcggtggcggtagcggtaccgattttaccctgaccatcagccgtctggagccggaagatagcgcggtgtactattgcctgcagtacggtagccatctgtatacctttggccaaggtaccaagctggaaattaaa

SEQ ID NO: 32 - DNA, 723 bp, synthetic construct
  misc_feature 1..723: note = Nucleotide sequence of the single-chain variable fragment (scFv) anti-sST2, variant A184Y
  V_region 1..348: note = Variable region of the heavy chain
  misc_feature 349..399: note = Linker
  V_region 400..723: note = Variable region of the light chain
  source 1..723: mol_type = other DNA
atgcaggtgcaactgcagcaatggggcgcgggtctgctgaaaccgagcgagaccctgagcctgacctgcgcggtttacggtggcagcttcagcggttactattggagctggattcgtcagccgccgggcaagggtctggagtggatcggcgaaattaaccacagcggtagcaccaactataacccgagcctgaaaagccgtgtgaccgttagcctggacaccagcaagaaccaattcagcctgaaactgagcagcgtgaccgcggcggacaccgcggtttactattgcgcgcgtggccgtggtctggtgcgttttgatagctggggccagggtaccaccgttaccgttagcagcggtggcggtggcagcggtggcggtggcagcggtggcggtggcagcgagattgttctgacccaaagcccgggtaccctgagcctgagcccgggtgatcgtgcgaccctgagctgccgtgcgagccagttcgtgggtagcagcagcgtggcgtggtaccagcaacgtccgggtcaagcgccgcgtctgctgatctattatgctgtgagccgtgcgagcggtattccggaccgtttcagcggtggcggtagcggtaccgattttaccctgaccatcagccgtctggagccggaagatagcgcggtgtactattgcctgcagtacggtagcgcgctgtatacctttggccaaggtaccaagctggaaattaaa

SEQ ID NO: 33 - protein, 116 aa, synthetic construct
  REGION 1..116: note = Variable domain of the heavy chain of the anti-sST2 antibody, variant E51Q
MQVQLQQWGAGLLKPSETLSLTCAVYGGSFSGYYWSWIRQPPGKGLEWIGQINHSGSTNYNPSLKSRVTVSLDTSKNQFSLKLSSVTAADTAVYYCARGRGLVRFDSWGQGTTVTV

SEQ ID NO: 34 - protein, 116 aa, synthetic construct
  REGION 1..116: note = Variable domain of the heavy chain of the anti-sST2 antibody, variant G101W
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRWLVRFDSWGQGTTVTV

SEQ ID NO: 35 - protein, 108 aa, synthetic construct
  REGION 1..108: note = Variable domain of the light chain of the anti-sST2 antibody, variant A228H/S166R
EIVLTQSPGTLSLSPGDRATLSCRASQFVGSSRVAWYQQRPGQAPRLLIYAAVSRASGIPDRFSGGGSGTDFTLTISRLEPEDSAVYYCLQYGSHLYTFGQGTKLEIK

SEQ ID NO: 36 - protein, 108 aa, synthetic construct
  REGION 1..108: note = Variable domain of the light chain of anti-sST2 antibody, variant A184Y
EIVLTQSPGTLSLSPGDRATLSCRASQFVGSSSVAWYQQRPGQAPRLLIYYAVSRASGIPDRFSGGGSGTDFTLTISRLEPEDSAVYYCLQYGSALYTFGQGTKLEIK

SEQ ID NO: 37 - protein, 215 aa, synthetic construct
  REGION 1..215: note = Variable and constant domains of the light chain of the anti-sST2 antibody
  DOMAIN 1..108: note = Variable domain of the light chain
  DOMAIN 109..215: note = Constant domain of the light chain (kappa), consensus sequence UNIPROT P01834-1, gen IGKC
EIVLTQSPGTLSLSPGDRATLSCRASQFVGSSSVAWYQQRPGQAPRLLIYAAVSRASGIPDRFSGGGSGTDFTLTISRLEPEDSAVYYCLQYGSALYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 645 bp, synthetic construct
  misc_feature 1..645: note = Nucleotide sequence of the variable and constant domain of the light chain of the anti-sST2 antibody
  V_region 1..324: note = Variable region of the light chain
  C_region 325..645: note = Constant region of the light chain (kappa)
  source 1..645: mol_type = other DNA
gagatcgtgctgacccagtctcctggcacactgtccctgagcccaggcgatagggccacactgagctgccgggcttctcagtttgtgggctccagctctgtggcctggtaccagcagagaccaggacaggctccaaggctgctgatctatgccgccgtgtccagggcctccggcatcccagaccggttctccggaggaggaagcggcaccgacttcaccctgacaatctccagactggagcctgaggacagcgccgtgtactattgcctgcagtacggctccgctctgtataccttcggccagggcacaaagctggagatcaagcgcaccgtggccgctccatccgtgttcatctttcccccttccgacgagcagctgaagtctggcacagcctccgtggtgtgcctgctgaacaacttctaccccagggaggccaaggtgcagtggaaggtggataacgctctgcagagcggcaattctcaggagtccgtgaccgagcaggacagcaaggattctacatattccctgtccagcaccctgacactgagcaaggccgattacgagaagcacaaggtgtatgcttgcgaggtgacccatcagggcctgtcttcccccgtgacaaagtctttcaacaggggcgagtgt

SEQ ID NO: 39 - protein, 446 aa, synthetic construct
  REGION 1..446: note = Variable and constant domains of the heavy chain of the anti-sST2 antibody
  DOMAIN 1..116: note = Variable domain of the heavy chain
  DOMAIN 117..446: note = Constant domain of the heavy chain of the anti-sST2 antibody. Consensus sequence UNIPROT P01857-1, gen IGHG1
MQVQLQQWGAGLLKPSETLSLTCAVYGGSFSGYYWSWIRQPPGKGLEWIGEINHSGSTNYNPSLKSRVTVSLDTSKNQFSLKLSSVTAADTAVYYCARGRGLVRFDSWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1338 bp, synthetic construct
  misc_feature 1..1338: note = Nucleotide sequence of the variable and constant domains of the heavy chain of the anti-sST2 antibody
  V_region 1..348: note = Variable region of the heavy chain
  C_region 349..1338: note = Constant region of the heavy chain of the anti-sST2 antibody. Consensus sequence UNIPROT P01857-1, gen IGHG1
  source 1..1338: mol_type = other DNA
atgcaggtgcagctgcagcagtggggagctggactgctgaagccatccgagaccctgagcctgacatgcgccgtgtacggcggcagcttttctggctactattggagctggatcagacagccacctggcaagggactggagtggatcggcgagatcaaccactccggcagcaccaactataatccttccctgaagagccgcgtgaccgtgtctctggacacatccaagaatcagttcagcctgaagctgtccagcgtgaccgccgctgacacagccgtgtactattgcgctaggggaaggggactggtgaggttcgattcttggggacagggaaccacagtgaccgtggccagcacaaagggcccttccgtgttccctctggctccatcttccaagtctacctccggaggaacagccgctctgggatgtctggtgaaggattacttccctgagccagtgaccgtgtcttggaactccggcgccctgacctctggagtgcatacatttcctgctgtgctgcagagctctggcctgtactccctgtccagcgtggtgaccgtgccatcttccagcctgggcacccagacatatatctgcaacgtgaatcacaagccttctaatacaaaggtggacaagaaggtggagccaaagtcctgtgataagacccatacatgcccaccatgtccagctcctgagctgctgggaggaccatccgtgttcctgtttcctccaaagcccaaggacaccctgatgatcagcaggaccccagaggtgacatgcgtggtggtggacgtgtctcacgaggatcccgaggtgaagtttaactggtacgtggatggcgtggaggtgcataatgctaagacaaagccaagagaggagcagtacaactccacctatcgcgtggtgagcgtgctgacagtgctgcaccaggactggctgaacggcaaggagtataagtgcaaggtgtccaataaggccctgccagctcccatcgagaagaccatcagcaaggctaagggacagccaagggagcctcaggtgtacacactgcccccttcccgggacgagctgaccaagaaccaggtgagcctgacatgtctggtgaagggcttctatcccagcgatatcgctgtggagtgggagtctaatggccagcctgagaacaattacaagaccacaccacccgtgctggacagcgatggctctttctttctgtattctaagctgaccgtggataagtcccggtggcagcagggcaacgtgtttagctgttctgtgatgcatgaggctctgcacaatcattacacacagaagtccctgagcctgtctcccggcaag

SEQ ID NO: 41 - protein, 17 aa, synthetic construct
  REGION 1..17: note = GS linker
SSGGGGSGGGGSGGGGS

SEQ ID NO: 42 - DNA, 51 bp, synthetic construct
  misc_feature 1..51: note = Nucleotide sequence of the GS linker
  source 1..51: mol_type = other DNA
agcagcggtggcggtggcagcggtggcggtggcagcggtggcggtggcagc

(19) United States
(12) Patent Application Publication
(10) Pub. No.: US 2026/0356960 A1
(43) Pub. Date: Nov. 12, 2026

(54) ANTI-HUMAN SST2 RECOMBINANT ANTIBODY, NUCLEIC ACID ENCODING THE SAME, AND PHARMACEUTICAL COMPOSITION

(71) Applicant: UNIVERSIDAD DE CHILE, Santiago (CL)

(72) Inventors: Marcela Alejandra HERMOSO RAMELLO, Santiago (CL); María Carmen MOLINA SAMPAYO, Los Andes (CL); Karen DUBOIS CAMACHO, Santiago (CL); Marjorie Katherine, Santiago (CL); Pedro Francisco NOVOA GUNDEL, Talcahuano (CL)

(21) Appl. No.: 19/489,536

(22) PCT Filed: Jun. 2, 2023
(86) PCT No.: PCT/IB2023/055712

§ 371 Date: Dec. 2, 2025

(51) Int. Cl. C07K 16/28

(52) U.S. Cl. CPC C07K 16/2866; C07K 2317/34; C07K 2317/565; C07K 2317/622

(57) ABSTRACT

The invention relates to an isolated antibody or a fragment thereof that binds to the SKECF peptide of the sST2 protein. In particular, the invention relates to a short variable fragment of an antibody (scFv) and to a human antibody with affinity for the sST2 molecule, to nucleic acids encoding the same, to the pharmaceutical compositions containing the same, and to the use thereof to treat inflammatory diseases, such as ulcerative colitis.

### TECHNICAL FIELD

The invention relates to an isolated antibody or a fragment thereof that binds to a SKECF peptide. In particular, the invention relates to a short variable fragment of an antibody (scFv) and to a human antibody with affinity for the SKECF peptide of the sST2 protein, to nucleic acids encoding the same, and to the pharmaceutical compositions containing the same.

### BACKGROUND OF THE INVENTION

Biological therapy plays a fundamental role in the control of inflammatory response, achieving fast and effective remission and its maintenance over time. It is indicated in patients with ulcerative colitis (UC) who are dependent on glucocorticoids or are resistant to this treatment and to immunomodulators (Simian, D., et al. (2013) Rol de infliximab en enfermedad inflamatoria intestinal: Experiencia local. Rev. Médica Chile 141, 1158-1165; Orlando, A. et al. (2011) The Italian Society of Gastroenterology (SIGE) and the Italian Group for the study of Inflammatory Bowel Disease (IG-IBD) Clinical Practice Guidelines: The use of tumor necrosis factor-alpha antagonist therapy in inflammatory bowel disease. Dig. Liver Dis. Off. J. Ital. Soc. Gastroenterol. Ital. Assoc. Study Liver 43, 1-20).

Currently, four biologic drugs corresponding to anti-TNFα antibodies (Infliximab, Adalimumab, and Golimumab) and anti-integrin α4β7 antibodies (Vedolizumab) have been tested (Simian, D., et al. (2013) Rol de infliximab en enfermedad inflamatoria intestinal: Experiencia local. Rev. Médica Chile 141, 1158-1165; Sandborn, W. J. et al. (2014) Subcutaneous golimumab induces clinical response and remission in patients with moderate-to-severe ulcerative colitis. Gastroenterology 146, 85-95; quiz e14-15; Singh, H., et al. (2016) Vedolizumab: A novel anti-integrin drug for treatment of inflammatory bowel disease. J. Nat. Sci. Biol. Med. 7, 4-9).

Infliximab is a chimeric monoclonal antibody directed against tumor necrosis factor TNF-α, which acts by binding to the membrane-bound and circulating portion, resulting in a cell-mediated cytotoxicity reaction and increased T4-lymphocyte-activated cell death. Therapy with Infliximab is effective in 53% to 65% of patients with ulcerative colitis (Orlando, A. et al. (2011) The Italian Society of Gastroenterology (SIGE) and the Italian Group for the study of Inflammatory Bowel Disease (IG-IBD) Clinical Practice Guidelines: The use of tumor necrosis factor-alpha antagonist therapy in inflammatory bowel disease. Dig. Liver Dis. Off. J. Ital. Soc. Gastroenterol. Ital. Assoc. Study Liver 43, 1-20; Danese, S., et al. (2013) Review article: the role of anti-TNF in the management of ulcerative colitis-past, present and future. Aliment. Pharmacol. Ther. 37, 855-866).

The antibody Vedolizumab, interacts with the 437 integrin preventing the binding of T lymphocytes to the endothelial surface of the gastrointestinal tract, preventing their extravasation to the affected tissue (Soler, D. et al. (2009) The binding specificity and selective antagonism of vedolizumab, an antialpha4beta7 integrin therapeutic antibody in development for inflammatory bowel diseases. J. Pharmacol. Exp. Ther. 330, 864-875). Vedolizumab has been found effective in the treatment of UC, however, therapies with these antibodies show systemic adverse effects associated with their use, among which are an increased risk of infectious diseases, in addition to the costs of treatment (Reddy, J. G. & Loftus, E. V. (2006) Safety of infliximab and other biologic agents in the inflammatory bowel diseases. Gastroenterol. Clin. North Am. 35, 837-855).

At present, and as a result of the surge in biologic therapy and personalized medicine, efforts have been focused on designing various strategies to neutralize a large number of potential therapeutic targets, most of them exploratory without satisfactory results.

Within the inflammatory component of UC there is a signaling pathway that could be used as a therapeutic target for this pathology, which comprises the IL-33/ST2 axis. IL-33 is a cytokine that binds to its ST2L receptor and promotes tissue and mucosal repair in the body. There is a soluble form of the ST2 gene, the sST2 protein, which binds to IL-33 preventing it from interacting with the ST2L receptor and inactivating the axis.

A growing number of evidence points to the relevance of the isoform sST2 in the development of a wide variety of pathologies, including cardiac diseases, such as myocardial fibrosis; inflammatory and autoimmune diseases, such as UC, asthma, and rheumatoid arthritis; pulmonary diseases; sepsis; hepatic and kidney diseases; and cancer (Homsak, E., & Gruson, D. (2020). Soluble ST2: A complex and diverse role in several diseases. Clinica chimica acta, 507, 75-87). In UC, patients show an increase in the content of the sST2 molecule at the intestinal mucosal level, which suggests that sST2 levels could contribute to the progression of the inflammatory process and that sST2 comprises a marker of inflammation (activity) in patients with this pathology (Díaz-Jiménez, D. et al. (2011) Soluble ST2: A new and promising activity marker in ulcerative colitis. World J. Gastroenterol. WJG 17, 2181-2190).

Thus, a greater understanding of intestinal mucosal homeostasis and the alterations in the local immune response that occur in these patients, along with the limitations and effectiveness of therapies currently in use, offers an opportunity to develop an alternative therapy that contemplates the intervention of the IL-33 axis. In particular, a therapy aimed at blocking sST2 considers an early or first-line target, which would recover intestinal barrier function by allowing activation of the IL-33/ST2L axis, ultimately leading to mucosal healing.

On the other hand, the ST2 protein isoforms, the ST2L receptor, and sST2, have a high sequence homology and differ only in 5 amino acids located at the carboxyl end of sST2. Commercially available antibodies (which are used to evaluate the levels of the molecule at laboratory level by techniques such as ELISA, Western Blot, or Immunofluorescence), recognize the total ST2 molecule without being able to differentiate the isoforms. Therefore, the development of a molecule that specifically recognizes the sST2 isoform, e.g., a therapeutic antibody (tAb), would be a novel strategy for the treatment or detection of this protein in pathologies where sST2 plays a pivotal role, such as inflammatory diseases like UC.

In addition to this background information, in recent years the number of biotechnological products used for the diagnosis and treatment of UC has increased. Among these, an important group of tAbs have allowed the development of therapies directed against selective targets, differentially expressed in greater quantities during severe inflammatory conditions, compared to control individuals. Eighty-five percent of biologic treatments and more than one third of all new agents currently under investigation are tAbs, such as the previously described anti-TNFα or anti α4β7. These complex molecules, of approximately 150 kDa, are bifunctional; on the one hand, they bind specifically to the target, usually aimed at blocking or neutralizing some molecule involved in the inflammatory process or the recruitment of inflammatory cells to the intestinal mucosa, and on the other hand, they activate effector functions characteristic of antibodies.

These antibodies, additionally, have low systemic toxicity and an appropriate half-life for prolonged action. TAbs are immunoglobulins (lg), in whose development recombinant DNA technology and genetic engineering is used, so they are generically called recombinant antibodies, rAbs.

Although the tAbs market is the fastest growing sector of the pharmaceutical industry, there is still a long way to go to reach a limit, mainly due to the wide variety of possible therapeutic targets that can be identified. In this sense, progress in the knowledge of the immunological mechanisms involved in the development of autoinflammatory and autoimmune pathologies is an opportunity to obtain new tAbs.

Regarding ulcerative colitis therapy, in particular antibodies that have been directed to the human ST2 molecule as a potential therapeutic target, several documents have been described.

For example, document AU 2020289777 A1 describes antibodies and antigen-binding fragments that specifically bind to human soluble ST2 protein. This document describes kits (ELISA) containing these antibodies, and methods for using these antibodies. Regarding the antibody, it is described that it is produced by the hybridoma PTA-10431 and that it can be used in ulcerative colitis, but the antibody is not evaluated in a valid model of this disease.

The document WO 2020/192266 A1, on the other hand, describes a kit for detecting the sST2 protein. Specifically, the invention relates to a latex-enhanced turbidimetric immunoassay kit for detecting the concentration and/or content of the sST2 in human samples. The kit can be applied to transmission light immunoturbidimetry and scattering light immunoturbidimetry. The kit comprises a buffer system, an anti-interference component, latex microspheres, an anti-sST2 antibody, and the like. The latex-enhanced immunoturbidimetric agent described by the invention can detect sST2 proteins within a range of 400 ng/ml and sensitivity can reach up to 0.1 ng/ml.

Document CN 111308098 describes a microfluidic fluorescence immunoassay chip for rapidly and quantitatively detecting sST2 in blood, belonging to the field of immunoassay. The chip comprises a tracing reagent comprising an sST2 monoclonal antibody marked by cyanine dye Cy5 and a quality control material, wherein the capture reagent contains an sST2 monoclonal antibody and a quality control material monoclonal antibody. According to the chip, an sST2 antigen in blood is marked by a tracer antibody to form an immune complex, and the immune complex is captured by a capture antibody and emits light under excitation of excitation light. The chip has higher sensitivity, specificity, and wider detection range, and can be used for evaluating the sST2 level of a patient and prompting the heart failure state.

On the other hand, document CN 110964106 A describes a preparation method of an anti-sST2 monoclonal antibody. The preparation method comprises the steps of preparation of sST2 recombinant protein, animal immunization, preparation of hybridoma cells, and preparation of a monoclonal antibody. The specific sST2 gene segment is selected, the conditions of a prokaryotic expression system are optimized, the expression quantity of protein and the purity of recombinant protein are improved; an immune mouse can generate an antibody with higher titer, and the prepared hybridoma cell strain can durably and efficiently secrete the high-performance anti-sST2 monoclonal antibody. The invention also describes an anti-sST2 monoclonal antibody, which is IgG1 subtype; and the antibody is good in consistency, has high titer, high immunocompetence, strong affinity, strong stability for detecting sST2, and high specificity and sensitivity. The invention also describes the application of the sST2 monoclonal antibody in immunodetection of sST2 products, and the sST2 monoclonal antibody has high detection specificity, high sensitivity and accuracy.

On the other hand, document CN 110208549 A describes a luminescent kit that detects sST2 protein expression, comprising a reagent R1, a reagent R2 and a magnetic separation reagent, wherein reagent R1 comprises a fluorescein isothiocyanate-labeled anti-human sST2 monoclonal antibody; the reagent R2 comprises an alkaline phosphatase-labeled anti-human sST2 monoclonal antibody; and the magnetic separation reagent comprises magnetic particles labeled with fluorescein isothiocyanate antibody.

Document CN 108181469 A describes a quantitative ELISA detection kit for sST2 and a method of preparation and application thereof. The object of the invention is to provide the ELISA quantitative detection kit with the advantages of a simple operation, shorter detection time, high precision and stable analysis performance. According to a schematics of the invention, the detection kit comprises an sST2 diluent, a wash solution, a detection antibody, a color developer solution, a stop buffer, a plurality of standard sST2 substances with different concentrations, an sST2 control substance and a microporous reaction plate, wherein the microporous reaction plate is coated with an sST2 monoclonal capture antibody and the detection antibody is a goat anti-human sST2 biotin monoclonal antibody. Neither the structure of the antibody, the epitope, nor its utility in the treatment of UC is mentioned.

Another document related to the invention is CN 107340386 A, which describes an sST2 detection kit based on microparticle chemical luminescence immunoassay, and relates to a chemical luminescence detection kit. The sST2 detection kit includes reaction buffer liquid, magnetic microparticles coated with monoclonal antibodies against sST2, monoclonal antibodies against sST2, luminescence liquid, a solution of sST2 antigen standard substance, and concentrated washing liquid. The reaction buffer liquid is prepared; the magnetic microparticles coated with the sST2 antibodies are prepared; the sST2 antibodies labeled with acridinium ester are prepared; the sST2 antigen standard substance solution is prepared; the luminescence liquid is prepared; the washing liquid is prepared. By adopting a microparticle chemical luminescence immunoassay technology, as compared to ELISA, it achieves higher sensitivity and stability, and fills a gap in the production of chemical luminescence diagnostic reagents for sST2 detection microparticles in human serum at home; the sST2 detection kit based on microparticle chemical luminescence immunoassay has the advantage that is easy to operate, has high sensitivity, is wide in linear range, shows stable results, is good in safety, is easy to automate and the like, and has a wide application perspective in the aspects of clinical examination and the like.

Document CN 105259353 A, on the other hand, describes a kit and method for detecting sST2 (soluble ST2) in blood from a patient with abdominal aortic aneurysm and/or aortic dissection. According to the kit and method, a pair of antibodies to identify different epitopes of sST2 is assembled to obtain a double-antibody sandwich ELISA and an immune colloidal gold test strip to perform quantitative and qualitative detection of sST2. Tests show that the ELISA kit and immune colloidal gold test strip have relatively high specificity and sensitivity on an abdominal aortic aneurysm marker: human sST2 protein, is easy to operate, can be used to detect abdominal aortic aneurysms, aortic dissection and other diseases in scientific research and clinical application, and has the functions of auxiliary diagnosis, guiding treatment and clinical prognosis.

Document CN 104151416 A, on the other hand, describes an anti-human sST2 monoclonal antibody and the application of the anti-human sST2 monoclonal antibody. A gene for encoding human sST2 protein is introduced into an appropriate Drosophila expression system by using a gene engineering method for the first time, and the recombinant human sST2 protein whose activity is very close to that of natural human sST2 protein is prepared. An antibody for sensitively identifying the natural sST2 protein in a human body can be obtained by immunizing animals by virtue of the protein so as to achieve the diagnosis or detection of relative diseases.

From the prior art described above, and to the best of the knowledge of the inventors, to date there is no antibody that is capable of differentiating between the ST2 protein isoforms, the ST2L receptor and sST2, which only differ in 5 amino acids located at the carboxyl end of sST2, known as the SKECF differentiating peptide. Consequently, there is a remaining necessity in the industry for an antibody that specifically binds to the sST2 protein, allowing its neutralization and ultimately activating the IL-33/ST2 axis for the restoration of cellular homeostasis.

### SUMMARY OF THE INVENTION

In order to meet the needs of the prior art, the present invention provides an isolated antibody or a fragment thereof that binds to a peptide having an amino acid sequence SKECF (SEQ ID NO: 1), which comprises:
a heavy-chain variable domain comprising:
   a CDR:H1 having an amino acid sequence as set forth in SEQ ID NO: 3;
   a CDR:H2 having an amino acid sequence as set forth in SEQ ID NO: 4; and
   a CDR:H3 having an amino acid sequence as set forth in SEQ ID NO: 5; and
a light-chain variable domain comprising:
   a CDR:L1 having an amino acid sequence as set forth in SEQ ID NO: 6;
   a CDR:L2 having an amino acid sequence as set forth in SEQ ID NO: 7; and
   a CDR:L3 having an amino acid sequence as set forth in SEQ ID NO: 8.

In one embodiment of the invention, the isolated antibody or fragment thereof binds to a peptide having an amino acid sequence SKECF, wherein said peptide is comprised within a protein or polypeptide. In a further embodiment, said protein is the Suppression of tumorigenicity-2 (ST2) protein or any of its variants or isoforms. In a yet further embodiment, said protein is the isoform sST2 of the ST2 protein. In a yet further embodiment, said sST2 protein is a mammalian sST2 protein. In a yet further embodiment, said mammalian sST2 protein is selected from the group of: human sST2 protein, bovine sST2 protein, porcine sST2 protein, ovine sST2 protein, caprine sST2 protein, or rodent sST2 protein. In a yet further embodiment of the invention, said mammalian sST2 protein is a human sST2 protein having an amino acid sequence as set forth in SEQ ID NO: 2.

In another embodiment of the invention, the isolated antibody or fragment thereof comprising a CDR:H2 having an amino acid sequence as set forth in SEQ ID NO: 4, further comprises a substitution of glycine (G) for tryptophan (W) at position 5.

In another embodiment of the invention, the isolated antibody or fragment thereof comprising a CDR:H3 having an amino acid sequence as set forth in SEQ ID NO: 5, further comprises a substitution of glutamic acid (E) for glutamine (Q) at position 1.

In another embodiment of the invention, the isolated antibody or fragment thereof comprises a heavy-chain variable domain comprising the sequence of SEQ ID NO: 9. In a further embodiment, said heavy-chain variable domain comprises at least one substitution in the amino acid sequence of SEQ ID NO: 9, selected from the group consisting of: glutamic acid (E) for glutamine (Q) at position 51, and glycine (G) for tryptophan (W) at position 101.

In another embodiment of the invention, the isolated antibody or fragment thereof comprising a CDR:L1 having an amino acid sequence as set forth in SEQ ID NO: 6, further comprises a substitution of serine(S) for arginine (R) at position 10.

In another embodiment of the invention, the isolated antibody or fragment thereof comprising a CDR:L2 having an amino acid sequence as set forth in SEQ ID NO: 7, further comprises a substitution of alanine (A) for tyrosine (Y) at position 1.

In another embodiment of the invention, the isolated antibody or fragment thereof comprising a CDR:L3 having an amino acid sequence as set forth in SEQ ID NO: 8, further comprises a substitution of alanine (A) for histidine (H) at position 6.

In another embodiment of the invention, the isolated antibody or fragment thereof further comprises:
a substitution of serine(S) for arginine (R) at position 10, in the CDR:L1 having an amino acid sequence as set forth in SEQ ID NO: 6; and,
a substitution of alanine (A) for histidine (H) at position 6, in the CDR:L3 having an amino acid sequence as set forth in SEQ ID NO: 8.

In another embodiment of the invention, the isolated antibody or fragment thereof comprises a light-chain variable domain comprising the sequence of SEQ ID NO: 10. In a further embodiment, said light-chain variable domain comprises at least one substitution in the amino acid sequence of SEQ ID NO: 10, selected from the group consisting of:
serine(S) for arginine (R) at position 33;
alanine (A) for tyrosine (Y) at position 51; and,
alanine (A) for histidine (H) at position 95.

In another embodiment, the antibody or a fragment thereof comprises an amino acid sequence selected from the group consisting of: SEQ ID NO: 11, SEQ ID NO: 12, SEQ ID NO: 13, SEQ ID NO: 14, or SEQ ID NO: 15.

In another embodiment, the isolated antibody or fragment thereof of the invention is a human IgG antibody. In a further embodiment, the isolated antibody or fragment thereof is a single-chain antibody fragment (scFv) of the heavy and light variable regions of a human IgG.

Another subject matter of the present invention is an isolated nucleic acid encoding for an isolated antibody or a fragment thereof as previously described.

In one embodiment, the isolated nucleic acid comprises a nucleotide sequence encoding for an isolated antibody or a fragment thereof, comprising:
a heavy-chain variable domain comprising:
   a CDR:H1 having an amino acid sequence as set forth in SEQ ID NO: 3;
   a CDR:H2 having an amino acid sequence as set forth in SEQ ID NO: 4; and
   a CDR:H3 having an amino acid sequence as set forth in SEQ ID NO: 5;
   and
a light-chain variable domain comprising:
   a CDR:L1 having an amino acid sequence as set forth in SEQ ID NO: 6;
   a CDR:L2 having an amino acid sequence as set forth in SEQ ID NO: 7; and
   a CDR:L3 having an amino acid sequence as set forth in SEQ ID NO: 8.

In a further embodiment, the isolated nucleic acid comprises:
a sequence encoding the heavy-chain variable domain comprising:
   a sequence encoding the CDR:H1, having a nucleotide sequence as set forth in SEQ ID NO: 16;
   a sequence encoding the CDR:H2, having a nucleotide sequence as set forth in SEQ ID NO: 17; and
   a sequence encoding the CDR:H3, having a nucleotide sequence as set forth in SEQ ID NO: 18;
   and,
a sequence encoding the light-chain variable domain comprising:
   a sequence encoding the CDR:L1, having a nucleotide sequence as set forth in SEQ ID NO: 19;
   a sequence encoding the CDR:L2, having a nucleotide sequence as set forth in SEQ ID NO: 20; and
   a sequence encoding the CDR:L3, having a nucleotide sequence as set forth in SEQ ID NO: 21.

In another embodiment, the isolated nucleic acid comprises a nucleotide sequence encoding for the heavy-chain variable domain, having an amino acid sequence as set forth in SEQ ID NO: 9, SEQ ID NO: 33, or SEQ ID NO: 34; and a nucleotide sequence encoding for the light-chain variable domain, having an amino acid sequence as set forth in SEQ ID NO: 10, SEQ ID NO: 35, or SEQ ID NO: 36. In a further embodiment, the isolated nucleic acid comprises a nucleotide sequence coding for the heavy-chain variable domain, selected from the group consisting of: SEQ ID NO: 22, SEQ ID NO: 23, and SEQ ID NO: 24; and a nucleotide sequence coding for the light-chain variable domain, selected from the group consisting of: SEQ ID NO: 25, SEQ ID NO: 26, and SEQ ID NO: 27.

In another embodiment, the isolated nucleic acid comprises a nucleotide sequence encoding for an isolated antibody, having an amino acid sequence as set forth in SEQ ID NO: 11, SEQ ID NO: 12, SEQ ID NO: 13, SEQ ID NO: 14, or SEQ ID NO: 15. In a further embodiment, the isolated nucleic acid comprises a nucleotide sequence selected from the group consisting of: SEQ ID NO: 28, SEQ ID NO: 29, SEQ ID NO: 30, SEQ ID NO: 31, or SEQ ID NO: 32

In another embodiment, the isolated nucleic acid as previously described comprises DNA, RNA or any of its derivatives.

Another subject matter of the present invention is a pharmaceutical composition, comprising a therapeutically effective amount of an antibody as described in the present application, together with a pharmaceutically acceptable excipient.

### BRIEF DESCRIPTION OF THE FIGURES

FIGS. 1A and 1B show a diagram of the mechanism of action of the complete human antibody AcHu-αsST2, which is subject matter of the present invention.

FIG. 2 depicts a single-chain scFv-type antibody (scFv-αsST2) (FIG. 2A) and a complete human antibody (AcHu-αsST2) (FIG. 2B) obtained by the present invention.

FIG. 3 shows the results of the ELISA assay to identify scFv-αsST2 clones recognizing the SKECF peptide of sST2.

FIG. 4 depicts a docking-type modeling between the sST2 protein and the scFv-αsST2 of the present invention. FIG. 4A shows the sST2 protein, FIG. 4B shows the scFv-αsST2, and FIG. 4C shows the interaction between the SKECF peptide of sST2 and scFv-αsST2.

FIG. 5 shows the model of complex 5 between sST2 and scFv-αsST2.

FIG. 6 shows the electrostatic surface map model between the scFv-αsST2 and sST2.

FIG. 7 shows the results of an immunoprecipitation analysis where it is observed that scFv-αsST2 recognizes the native protein of sST2. Loading order: lane 1 is immunoprecipitation (IPP) of supernatant (SN) of HMC-1 (with scFv+αHA), lane 2 is Ladder full range. Developing:anti ST2 goat polyclonal (1:250), anti-goat HRP (1:5000).

FIG. 8 shows the detection of native sST2 by scFv-αsST2 in HMC-1 cells.

FIG. 9 shows the results of the evaluation of the dissociation constant of scFv-αsST2 binding to sST2 by ELISA.

FIG. 10 shows the results of the affinity study between scFv-αsST2 and SKECF differentiating peptide of sST2 by Surface Plasmon Resonance. FIG. 10A shows a sensogram with the Resonance units (RU) measured for different concentrations of scFv-αsST2 where each curve represents the stages of association and dissociation between molecules. FIG. 10B shows a curve of RU vs concentration and the linear regression calculation from which the value of the dissociation constant (K_{D}) was obtained.

FIG. 11 shows the chromatogram of the purification of AcHu-αsST2.

FIG. 12 shows the results of ex vivo assays of AcHu-αsST2, showing that this antibody decreases TNF levels by increasing IL-10 levels in explants from patients with ulcerative colitis. FIG. 12A shows that AcHu-αsST2 (ACHU) significantly decreases TNF levels compared to control (C). FIG. 12B shows that AcHu-αsST2 (ACHU) significantly decreases IL-10 levels compared to control (C).

FIG. 13 shows the structural model of the protein-protein interaction complex: sST2 on the left with its SKECF epitope at the center of the figure and scFv-αsST2 on the right. Also, CDRs are highlighted at the center, where the heavy-chain CDRs: CDR:H1, CDR:H2 and CDR:H3 are below, and the light-chain CDRs: CDR:L1, CDR:L2 and CDR:L3 are at the top.

FIG. 14 shows the binding energies values (ΔG) of mutant variants obtained by site-directed mutation of scFv-αsST2.

FIG. 15 shows the procedure for affinity determination by surface plasmon resonance. The biotinylated peptide (representative of sST2) bound to the sensor surface interacts with scFv molecules contained in the microfluid.

FIG. 16 shows the results of affinity determination of scFv-αsST2 and its mutant variants obtained by site-directed mutation.

### DETAILED DESCRIPTION OF THE INVENTION

The present invention is directed to a biopharmaceutical, in particular to an antibody or a fragment thereof directed against the sST2 receptor, the neutralization of which would enable the effector functions of IL-33. As previously described, the ST2/IL-33 axis has been found to be relevant in several pathologies, which include but are not limited to inflammatory, cancer, and cardiac diseases. In the context of UC, the binding of the antibody to sST2 ultimately stimulates the repair of the (mucosal) epithelium and the activation of different cell types, contributing to the balance of the immune response in the intestinal mucosa of patients with UC.

Notwithstanding the evidence provided herein, the therapeutic potential of the antibodies, nucleic acids, and pharmaceutical compositions of the present invention is not limited to UC.

All technical and scientific terms used to describe the present invention have the same meaning understood to a person with basic knowledge in the technical field in question. However, in order to define the scope of the invention more clearly, a list of the terminology used in this description is included below.

The term “antibody” should be understood as a glycoprotein belonging to the superfamily of immunoglobulins, which specifically binds, or is immunologically reactive to, a particular antigen. Antibodies recognize a specific region of said antigen (epitope) through a variable Fab region (antigen-binding fragment), and the antibody's ability to communicate with components of the immune system is through its Fc region (fragment crystallizable region). Antibodies consist of a subunit called the heavy chain comprising a variable region (V_{H}) and several constant regions (C_{H}1, C_{H}2, etc.), and a subunit called the light chain comprising a variable region (V_{L}) and a constant region (C_{L}). The variable regions comprise complementarity determining regions (CDRs), there being three CDRs in the heavy chain (CDR:H1, CDR:H2, and CDR:H3), and three CDRs in the light chain (CDR:L1, CDR:L2, and CDR:L3). CDRs are the regions that bind to an epitope of a specific antigen, and a set of CDRs constitutes a paratope, which is the specific binding site of the antibody to the epitope of its corresponding antigen. Antibodies are classified according to the type of heavy chain they have, within the classes IgM, IgG, IgA, IgD, IgE, where the heavy chains mu (μ) corresponds to class IgM, gamma (γ) to IgG, alpha (α) to IgA, delta (δ) to IgD, and epsilon (ε) to IgE. In addition, antibodies comprise varied three-dimensional structures such as, for example, but are not limited to, monoclonal antibodies, polyclonal antibodies, genetically modified antibodies, chimeric antibodies, multispecific antibodies (e.g., bispecific antibodies, diabodies, triabodies, tetrabodies), antibody fragments that retain their antigen binding activity such as, for example, Fab′, F(ab′)₂, Fab, Fv, rIgG, and scFv.

The term “single-chain antibody” or “scFv” should be understood as a fusion protein in which the variable regions of the heavy chain (V_{H}) and light chain (V_{L}) of a traditional antibody are bound through a linker sequence to form a single polypeptide chain.

The term “nucleotide sequence” should be understood as a double strand of DNA, or a single strand of DNA, natural or synthetic, or products of the transcription of said DNA (for example, RNA molecules). It should be understood that the present invention does not relate to genomic nucleotide sequences in their natural state, but refers to nucleotide sequences in an isolated, or purified, or partially purified state, or obtained by synthetic or recombinant means, employing any genetic engineering method known in the state of the art.

The term “amino acid sequence” should be understood as an amino acid sequence, natural or synthetic, or products of RNA translation. When these polypeptides have a stable, three-dimensional structure they are called proteins. It should be understood that the present invention does not relate to amino acid sequences in their natural state, but refers to amino acid or protein sequences in an isolated, or purified, or partially purified state, or obtained by synthetic or recombinant means, employing any genetic engineering method known in the state of the art.

The term “substitution” should be understood as the change of a determined sequence at a particular position. By way of example, a substitution at a particular position of an amino acid sequence for a determined amino acid means that, at that position, instead of the amino acid that was originally part of the sequence, there is now the amino acid with which it was substituted. This amino acid substitution can be caused, for example, by a modification in the nucleotide sequence from which the amino acids are translated. An antibody, peptide, or protein comprising a substitution will be interchangeably referred to as a “mutant”.

In this context, the term “E51Q” should be understood as an isolated antibody or fragment thereof comprising a substitution of glutamic acid (E) for glutamine (Q) at position 1 of SEQ ID NO: 4, or equivalently, a substitution of glutamic acid (E) for glutamine (Q) at position 51 of SEQ ID NO: 9.

The term “A228H/S166R” should be understood as an isolated antibody or a fragment thereof comprising a substitution of serine(S) for arginine (R) at position 10 of SEQ ID NO: 6, or a substitution of serine(S) for arginine (R) at position 33 of SEQ ID NO: 10. While at the same time comprising a substitution of alanine (A) for histidine (H) at position 6 of SEQ ID NO: 8, or a substitution of alanine (A) for histidine (H) at position 95 of SEQ ID NO: 10.

The term “A184Y” should be understood as an isolated antibody or fragment thereof comprising a substitution of alanine (A) for tyrosine (Y) at position 1 of SEQ ID NO: 7, or comprising a substitution of alanine (A) for tyrosine (Y) at position 51 of SEQ ID NO: 10.

The term “G101W” should be understood as an isolated antibody or fragment thereof comprising a substitution of glycine (G) for tryptophan (W) at position 5 of SEQ ID NO: 5, or comprising a substitution of glycine (G) for tryptophan (W) at position 101 of SEQ ID NO: 9.

The term “identity” between nucleotide or amino acid sequences should be understood as the percentage of identical nucleotides or amino acids that the compared sequences share with each other, in a particular window of comparison. The percentage of identity can be calculated using a sequence comparison algorithm or by manual alignment and visual inspection. For example, sequences and identity percentages can be obtained using computer resources available on the Internet such as the Basic Local Alignment Search Tool (BLAST) from the U.S. NIH, or the computer program FastDB. Nucleotide sequence identity can also be determined by nucleotide sequence hybridization assays. The higher the degrees of hybridization astringency used in the assay, the greater the sequence complementarity required for the sequences to hybridize. High astringency conditions are described by Sambrook et al. (Molecular Cloning A Laboratory Manual, Cold Spring Harbor Press, 1989).

The term “flexible peptide” should be understood as an amino acid sequence artificially created by genetic engineering tools, which allows the creation of a suitable bond between amino acid sequences functioning as a linker sequence, for example, between two subunits of a protein and which allows the creation of a single amino acid sequence that includes both subunits. This linker sequence allows said protein to fold correctly, without losing the functionality of its subunits.

In the present invention, the flexible peptide corresponds to SEQ ID NO: 41, which is located from position 116 to 133 of SEQ ID NO: 11, SEQ ID NO.: 12, SEQ ID NO.: 13, SEQ ID NO.: 14, and SEQ ID NO.: 15. However, other flexible peptide sequences are valid for the same purpose, as described by Chen et al. (Chen, X., Zaro, J. L., & Shen, W.-C. (2013). Fusion protein linkers: Property, design and functionality. Advanced Drug Delivery Reviews, 65(10), 1357-1369. doi:10.1016/j.addr.2012.09.039).

The term “biopharmaceutical” should be understood as a drug that is produced in genetically modified organisms, either transgenic organisms or those modified only in a particular tissue. Particularly, the term “biopharmaceutical” is used interchangeably with “pharmaceutical composition”, both referring to a drug having a therapeutic effect in the prevention, amelioration, or treatment of a specific disease or diseases.

In order to meet the needs of the prior art, the present invention provides an isolated antibody or a fragment thereof that binds to a peptide having an amino acid sequence SKECF (SEQ ID NO: 1), which comprises:
a heavy-chain variable domain comprising:
   a CDR:H1 having an amino acid sequence as set forth in SEQ ID NO: 3;
   a CDR:H2 having an amino acid sequence as set forth in SEQ ID NO: 4; and
   a CDR:H3 having an amino acid sequence as set forth in SEQ ID NO: 5; and
a light-chain variable domain comprising:
   a CDR:L1 having an amino acid sequence as set forth in SEQ ID NO: 6;
   a CDR:L2 having an amino acid sequence as set forth in SEQ ID NO: 7; and
   a CDR:L3 having an amino acid sequence as set forth in SEQ ID NO: 8.

In one embodiment of the invention, the isolated antibody or fragment thereof binds to a peptide having an amino acid sequence SKECF, wherein said peptide is comprised within a protein or polypeptide. In a further embodiment, said protein is the Suppression of tumorigenicity-2 (ST2) protein or any of its variants or isoforms. In a yet further embodiment, said protein is the isoform sST2 of the ST2 protein. In a yet further embodiment, said sST2 protein is a mammalian sST2 protein. In a yet further embodiment, said mammalian sST2 protein is selected from the group of: human sST2 protein, bovine sST2 protein, porcine sST2 protein, ovine sST2 protein, caprine sST2 protein, or rodent sST2 protein. In a yet further embodiment of the invention, said mammalian sST2 protein is a human sST2 protein having an amino acid sequence as set forth in SEQ ID NO: 2.

In another embodiment of the invention, the isolated antibody or fragment thereof comprising a CDR:H2 having an amino acid sequence as set forth in SEQ ID NO: 4, further comprises a substitution of glycine (G) for tryptophan (W) at position 5.

In another embodiment of the invention, the isolated antibody or fragment thereof comprising a CDR:H3 having an amino acid sequence as set forth in SEQ ID NO: 5, further comprises a substitution of glutamic acid (E) for glutamine (Q) at position 1.

In another embodiment of the invention, the isolated antibody or fragment thereof comprises a heavy-chain variable domain comprising the sequence of SEQ ID NO: 9. In a further embodiment, said heavy-chain variable domain comprises at least one substitution in the amino acid sequence of SEQ ID NO: 9, selected from the group consisting of: glutamic acid (E) for glutamine (Q) at position 51, and glycine (G) for tryptophan (W) at position 101.

In another embodiment of the invention, the isolated antibody or fragment thereof comprising a CDR:L1 having an amino acid sequence as set forth in SEQ ID NO: 6, further comprises a substitution of serine(S) for arginine (R) at position 10.

In another embodiment of the invention, the isolated antibody or fragment thereof comprising a CDR:L2 having an amino acid sequence as set forth in SEQ ID NO: 7, further comprises a substitution of alanine (A) for tyrosine (Y) at position 1.

In another embodiment of the invention, the isolated antibody or fragment thereof comprising a CDR:L3 having an amino acid sequence as set forth in SEQ ID NO: 8, further comprises a substitution of alanine (A) for histidine (H) at position 6.

In another embodiment of the invention, the isolated antibody or fragment thereof further comprises:
a substitution of serine(S) for arginine (R) at position 10, in the CDR:L1 having an amino acid sequence as set forth in SEQ ID NO: 6; and,
a substitution of alanine (A) for histidine (H) at position 6, in the CDR:L3 having an amino acid sequence as set forth in SEQ ID NO: 8.

In another embodiment of the invention, the isolated antibody or fragment thereof comprises a light-chain variable domain comprising the sequence of SEQ ID NO: 10. In a further embodiment, said light-chain variable domain comprises at least one substitution in the amino acid sequence of SEQ ID NO: 10, selected from the group consisting of:
serine(S) for arginine (R) at position 33;
alanine (A) for tyrosine (Y) at position 51; and,
alanine (A) for histidine (H) at position 95.

In another embodiment, the antibody or a fragment thereof comprises an amino acid sequence selected form the group consisting of: SEQ ID NO: 11, SEQ ID NO: 12, SEQ ID NO: 13, SEQ ID NO: 14, or SEQ ID NO: 15. These correspond to the antibodies; scFv without mutation, scFv E51Q, scFv G101W, scFv A228H/S166R, and scFv A184Y, respectively.

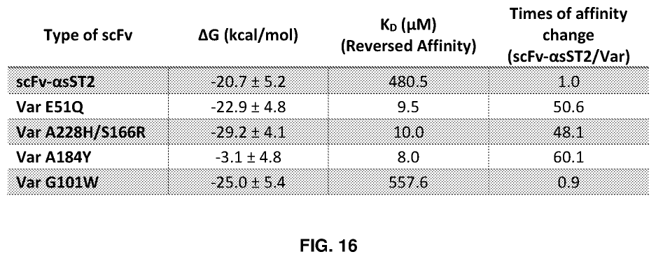

The various options of light and or heavy chain sequence substitutions correspond to isolated mutant antibodies or fragments thereof with equal or higher affinity for the SKECF peptide (SEQ ID NO: 1) of the sST2 protein (SEQ ID NO: 2), as compared to the antibody comprising the sequence SEQ ID NO: 11. In this manner, the various substitutions increase the affinity for SKECF (SEQ ID NO: 1) of one or more of the three CDRs in its light-chain variable domain or one or more of the three CDRs in its heavy-chain variable domain. The affinity increase for each substitution can be seen in FIG. 16.

In another embodiment, the isolated antibody or fragment thereof of the invention is a human IgG antibody. In a further embodiment, the isolated antibody or fragment thereof is a single-chain antibody fragment (scFv) of the heavy and light variable regions of a human IgG.

Another subject matter of the present invention is an isolated nucleic acid encoding for an isolated antibody or a fragment thereof as previously described.

In one embodiment, the isolated nucleic acid comprises a nucleotide sequence encoding for an isolated antibody or a fragment thereof, comprising:
a heavy-chain variable domain comprising:
   a CDR:H1 having an amino acid sequence as set forth in SEQ ID NO: 3;
   a CDR:H2 having an amino acid sequence as set forth in SEQ ID NO: 4; and
   a CDR:H3 having an amino acid sequence as set forth in SEQ ID NO: 5;
   and
a light-chain variable domain comprising:
   a CDR:L1 having an amino acid sequence as set forth in SEQ ID NO: 6;
   a CDR:L2 having an amino acid sequence as set forth in SEQ ID NO: 7; and
   a CDR:L3 having an amino acid sequence as set forth in SEQ ID NO: 8.

In a further embodiment, the isolated nucleic acid comprises:
a sequence encoding the heavy-chain variable domain comprising:
   a sequence encoding the CDR:H1, having a nucleotide sequence as set forth in SEQ ID NO: 16;
   a sequence encoding the CDR:H2, having a nucleotide sequence as set forth in SEQ ID NO: 17; and
   a sequence encoding the CDR:H3, having a nucleotide sequence as set forth in SEQ ID NO: 18;
   and,
a sequence encoding the light-chain variable domain comprising:
   a sequence encoding the CDR:L1, having a nucleotide sequence as set forth in SEQ ID NO: 19;
   a sequence encoding the CDR:L2, having a nucleotide sequence as set forth in SEQ ID NO: 20; and
   a sequence encoding the CDR:L3, having a nucleotide sequence as set forth in SEQ ID NO: 21.

In another embodiment, the isolated nucleic acid comprises a nucleotide sequence encoding for the heavy-chain variable domain, having an amino acid sequence as set forth in SEQ ID NO: 9, SEQ ID NO: 33, or SEQ ID NO: 34; and a nucleotide sequence encoding for the light-chain variable domain, having an amino acid sequence as set forth in SEQ ID NO: 10, SEQ ID NO: 35, or SEQ ID NO: 36. In a further embodiment, the isolated nucleic acid comprises a nucleotide sequence coding for the heavy-chain variable domain, selected from the group consisting of: SEQ ID NO: 22, SEQ ID NO: 23, and SEQ ID NO: 24; and a nucleotide sequence coding for the light-chain variable domain, selected from the group consisting of: SEQ ID NO: 25, SEQ ID NO: 26, and SEQ ID NO: 27.

In another embodiment, the isolated nucleic acid comprises a nucleotide sequence encoding for an isolated antibody, having an amino acid sequence as set forth in SEQ ID NO: 11, SEQ ID NO: 12, SEQ ID NO: 13, SEQ ID NO: 14, or SEQ ID NO: 15. In a further embodiment, the isolated nucleic acid comprises a nucleotide sequence selected from the group consisting of: SEQ ID NO: 28, SEQ ID NO: 29, SEQ ID NO: 30, SEQ ID NO: 31, or SEQ ID NO: 32

In another embodiment, the isolated nucleic acid as previously described comprises DNA, RNA or any of its derivatives.

Another subject matter of the present invention is a pharmaceutical composition, comprising a therapeutically effective amount of an antibody as described in the present application, together with a pharmaceutically acceptable excipient.

Acceptable carriers, excipients, or stabilizers are non-toxic to the recipients of the dosages and concentrations employed and may comprise buffer solutions such as phosphate, citrate and other organic acids; salts such as sodium chloride; antioxidants including ascorbic acid and methionine; preservatives (such as octadecyl dimethyl benzyl ammonium chloride; hexamethonium chloride; benzalkonium chloride, benzethonium chloride; phenol, butyl or benzyl alcohol; alkyl parabens, such as methyl or propylparaben; catechol; resorcinol; cyclohexanol; 3-pentanol; and mcresol); low molecular weight polypeptides (less than approximately 10 residues); proteins, such as serum albumin, gelatin or Ig; hydrophilic polymers such as polyvinylpyrrolidone; amino acids such as glycine, glutamine, asparagine, histidine, arginine or lysine; monosaccharides, disaccharides and other carbohydrates including glucose, mannose or dextrins; chelating agents such as EDTA; sugars such as sucrose, mannitol, trehalose or sorbitol; salt-forming counterions such as sodium; metal complexes (e.g., Zn-protein complexes); and/or nonionic surfactants such as TWEEN, PLURONICS or polyethylene glycol (PEG).

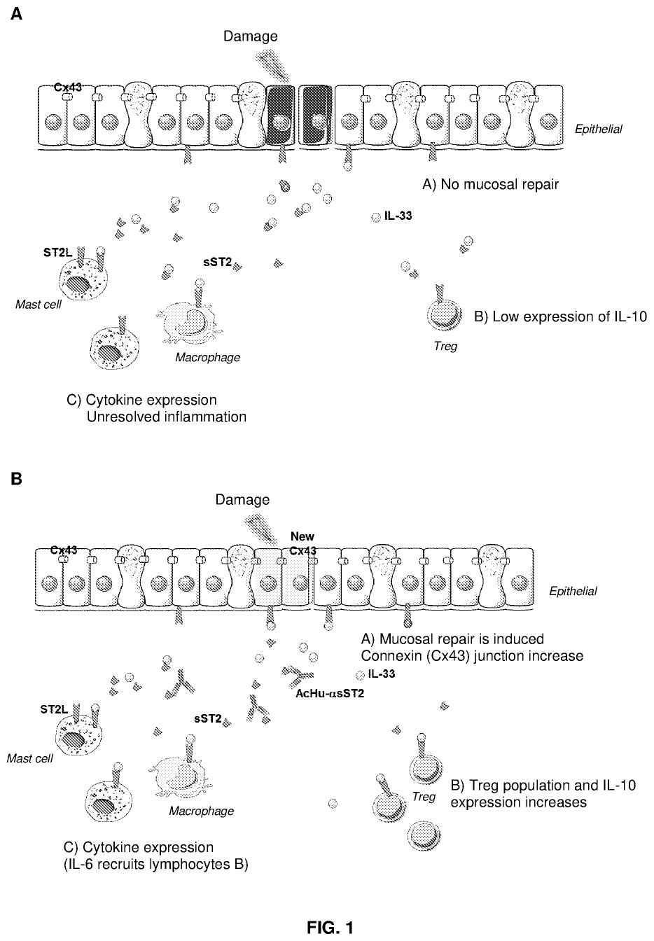

FIG. 1A depicts a situation of an inflammatory process without the intervention of the therapeutic AcHu-αsST2 antibody. The sST2 is a bait receptor of IL-33 that shares much of its sequence with the extracellular domain of the ubiquitous ST2L receptor, anchored to the plasma membrane, whose recognition leads to cell activation (mast cells, CD4+, Th2, Treg, monocytes, macrophages, dendritic, Tγδ, epithelial, among others), and promotes inflammatory and tissue regeneration responses (mucosal healing, in the case of the intestine). In inflammatory processes of the intestinal mucosa, such as in ulcerative colitis (UC), sST2 levels are increased as is the release of IL-33, due to tissue damage (epithelial barrier).

FIG. 1B shows a situation of the same process with the intervention of the AcHu-αsST2 antibody. The AcHu-αsST2 binds to sST2 molecules, neutralizing them and, as a consequence, freeing IL-33 by increasing its availability to bind to the ST2L receptor expressed by different mucosal resident and infiltrating cell types (including regulatory T lymphocytes), and thus facilitating its effects that lead to the restoration of intestinal homeostasis and tissue repair.

To test the hypothesis of the application of the antibody of the invention in inflammatory processes, and in particular its usefulness in the treatment of UC, an scFv-type (single-chain variable fragment) Antibody (Ab) was selected; a potentially neutralizing single-chain fragment Ab of the heavy and light variable regions, which recognizes the sST2 receptor and does not cross-react with the other variant of ST2 (ST2L).

Using recombinant DNA technology and genetic engineering, the heavy- and light-chain domains of immunoglobulins (Igs) are bound by a flexible peptide, generating a scFv, which is the basis for the production of a complete human antibody (AcHu).

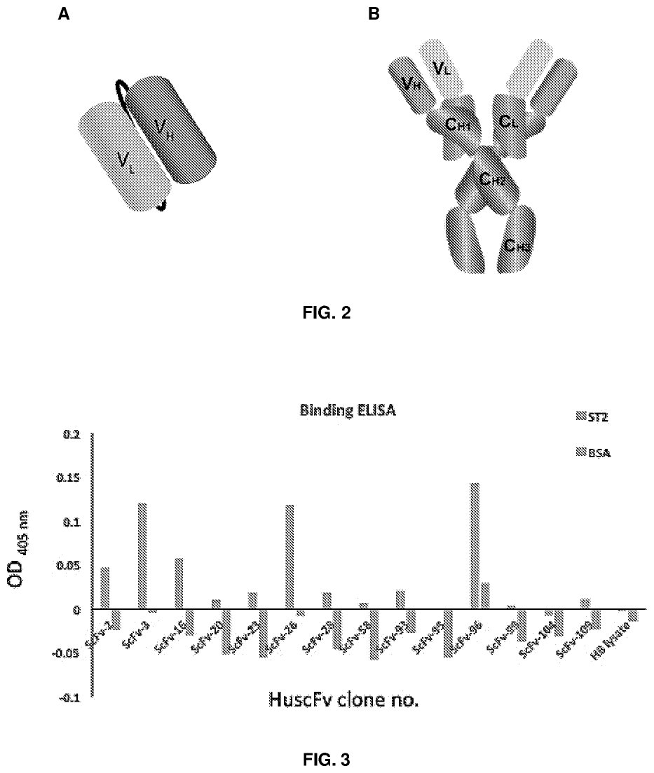

FIG. 2 shows the single-chain scFv-type antibody that is the object of the invention and the complete human antibody obtained from the scFv. As observed in FIG. 2A, the scFv or anti-sST2 antibody fragment is formed by heavy and light variable regions, V_{H} and V_{L}, bound in a single linear chain by a flexible peptide. The sequence of the flexible peptide corresponds to SEQ ID NO: 41.

FIG. 2B shows the antibody, or also referred to as AcHu αsST2, corresponding to an anti-sST2 immunoglobulin, which is a protein consisting of four chains, two of larger size referred to as heavy chains (H) and two of smaller size or light chains (L). Each L chain is bound to an H chain and both H chains are bound to each other through disulfide bonds. At the amino terminal end of each chain are the V_{H} and V_{L} variable domains of the heavy and light chain, respectively, which in this case do not contain the flexible peptide as seen in the structure of FIG. 2A. The combination of a V_{H} domain with another V_{L} domain forms the antigen-binding site with high specificity, in this case the therapeutic target sST2. The V_{H} and V_{L} domains originate from the anti-sST2 scFv previously generated in the laboratory. Each antibody has two variable domains (V_{H} and V_{L}), making it a bivalent molecule, while on the opposite side of the molecule is the Fc region, responsible for the effector functions of immunity, such as promoting immunocomplex formation and allowing clearance of sST2.

In the laboratory of Dr. Chaicumpa (Mahidol University, Thailand), a library of filamentous phages expressing human scFv on their surface was generated from healthy donor cells. From this library, a scFv directed against sST2 (scFv-αsST2) was selected.

For this selection, the differentiating or exclusive peptide of the sST2 receptor sequence (SKECF peptide as shown in SEQ ID NO: 1) was synthesized and used to select the specific scFv recognizing this peptide by ELISA assays. The selected clone, scFv-αsST2, was used to evaluate affinity against sST2, and its sequence was used to develop a fully human neutralizing antibody AcHu-αsST2, which recognizes sST2, and potentially reverses inflammation in preliminary ex vivo ulcerative colitis (UC) assays.

Additionally, theoretical values for the Gibbs free energy (ΔG) were obtained through in silico studies from different scFv-αsST2 mutants comprising substitutions that could favor electrostatic interactions with sST2. In this way, different mutant antibodies were synthesized and then evaluated in relation to their affinity for the SKECF peptide, and showed an increase in affinity compared to the unmutated antibody.

The AcHu-αsST2 and scFv-αsST2 generated from the invention are biotechnological products with potential for use in the treatment of UC and also other diseases where a progressive and chronic increase of the sST2 variant is observed, such as inflammatory, cancer, and cardiac diseases.

These biopharmaceuticals could also be used in selective adjunctive therapy to other therapies to treat UC, increasing the effectiveness of other drugs directed against different therapeutic targets, or when patients are resistant to these drugs, such as anti-TNF or anti-integrin, which have been reported to be around 20% of patients.

As it has been described, AcHu-αsST2 is a recombinant antibody whose mechanism of pharmacological action is to bind to the sST2 molecule present in the extracellular environment, blocking its binding to IL-33 and allowing the restoring effect of IL-33 through its binding to the ST2L receptor. Additionally, the binding of AcHu-αsST2 to the sST2 molecule enables the formation of immunocomplexes which are phagocytosed by cells of the immune system and cleared from the extracellular environment (clearance effect).

As a result of the present invention, recombinant antibodies were obtained that proved to be of pharmacological interest in ex vivo assays with colonoscopy biopsies from UC patients, and in controls and in vitro assays using a human mast cell line (HMC-1) where their effectiveness and specificity and effectiveness, respectively, were evaluated.

In a study performed in the laboratory, it was observed that UC patients with higher levels of sST2 have a higher risk of requiring surgery for the control of their pathology; therefore, these antibodies would have an additional beneficial therapeutic impact, greater than other therapies, by avoiding the need for surgery.

An important aspect of the possible application of the antibodies that are the object of the present invention, is that there are no options for the early treatment of the disease, and in particular the AcHu-αsST2 could be used as first line therapy, in which molecular components of the early innate immune response intervene, thus preventing lesions in the digestive tract from being deeper, and consequently, preventing the complications that derive from it, such as lower digestive hemorrhage, fulminant colitis, and even risk of developing colorectal cancer. Moreover, when administered early, this treatment would make it possible to reduce or interrupt the administration of corticosteroids in accordance with standard treatments.

### EXAMPLES OF EMBODIMENTS

The following examples are intended to illustrate the invention and its preferred embodiments, but under no circumstances should they be considered to restrict the scope of the invention, which will be defined by the scope of the claims appended hereto.

### Example 1: Selection and Characterization of a Viral Particle Expressing scFv-αsST2 (scFv-αsST2-phage)

In the laboratory of Dr. Chaicumpa (Mahidol University, Thailand), a library of filamentous phages expressing human scFv on their surface was generated from healthy donor cells. From said library, the inventors selected a scFv directed against sST2 (scFv-αsST2) by sequentially performing the following steps described below:
1.—Selecting a peptide from the sST2 molecule to which the recombinant antibody binds;
2.—Selecting and characterizing a viral particle expressing scFv-αsST2 (scFv-αsST2-phage).

The selection and characterization of the anti-sST2 scFv was performed by recombinant DNA technology and genetic engineering using a library of filamentous phages (Phage M13) expressing scFvs of human origin on their surface, from a repertoire of variable light and heavy chains from 60 healthy donors. Selection of the viral particle expressing anti-sST2 scFv (scFv-αsST2-phage) was performed through 3 cycles of “panning” with the differentiating peptide: SKECF from SEQ ID NO: 1.

Subsequently, 33 clones were randomly selected and an ELISA assay was performed, using plates sensitized with the sST2-SKECF peptide. The clones with the highest signal (number 3 and 96) were selected (FIG. 3). The two clones with the highest reactivity against the selected SKECF peptide were amplified and the segments encoding scFv were sequenced. These had a similar sequence, so they will be used for further molecular generation. The fact that the selected clones had a similar sequence is suggestive of an appropriate selection since this is explained by the enrichment of a scFv-αST2 by competition. The scFvs 3 and 96 were produced in E. coli BL21 (DE3) bacteria and purified using a nickel matrix. Because the production efficiency was higher for scFv 96 (approximately 1 mg protein), all in silico assays and in vitro specificity and interaction assays between scFv and sST2 were performed with scFv 96.

For this selection, the differentiating or exclusive peptide of the sST2 receptor sequence (SKECF as shown in SEQ ID NO: 1) was synthesized and used to select the specific scFv recognizing this peptide by ELISA assays. The selected clone, scFv-αsST2, was used to assess affinity against sST2 and its sequence was used to develop a fully neutralizing human antibody AcHu-αsST2, which recognizes sST2 and potentially reverses inflammation in preliminary ex vivo ulcerative colitis assays.

### Example 2: Study of the Interaction Model Between the scFv and the sST2 Protein

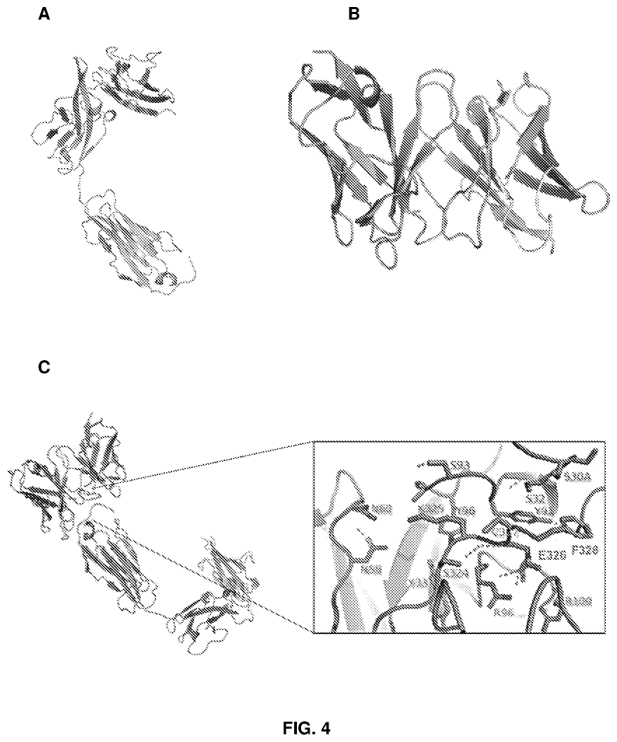

To study the interaction model between the scFv and the sST2 protein, an in silico assay was performed consisting of a molecular docking study followed by a molecular dynamics study to know the relevant residues in the interaction zone of both proteins. Using the crystal structure of the membrane protein ST2L: PDB 4KC3 14, homology modeling of the sST2 protein was performed on the I-TASSER 15 platform (Yang, J., & Zhang, Y. (2015). Protein Structure and Function Prediction Using I-TASSER. Current Protocols in Bioinformatics, 5.8.1-5.8.15.) which was also used to obtain the scFv model from its amino acid sequence (FIG. 4). FIG. 4A depicts the model of the sST2 structure: the protein structure of sST2 is depicted in green (major structure), with the scFv 96-1 epitope at the carboxyl end highlighted in yellow (tail located in the lower left area). FIG. 4B depicts the modeling of scFv 96-1:light chain variable (V_{L}) in cyan (upper left), heavy chain variable (V_{H}) in green (upper right). The complementarity determining regions (CDRs) corresponding to CDRL1-3 and CDRH1-3 are colored in blue (lower left) and yellow (lower right), respectively. FIG. 4C shows the structure of the sST2/scFv 96-1 complex on the left, and a close-up of the model at the scFv-differentiating peptide sST2 interaction site on the right. The representation of the structures is shown in green (lower right) for sST2 and purple (upper right) and cyan (upper left) for scFv 96-1 V_{L} and V_{H}, respectively. The two structural models were optimized using ModRefiner 16 (Xu, D., & Zhang, Y. (2011). Improving the Physical Realism and Structural Accuracy of Protein Models by a Two-Step Atomic-Level Energy Minimization. Biophysical Journal, 101(10), 2525-2534) prior to the molecular docking study. In the platform Clus Pro 2.0 17, in its antibody study mode (Kozakov, D., Hall, D. R., Xia, B., Porter, K. A., Padhorny, D., Yueh, C., . . . Vajda, S. (2017). The ClusPro web server for protein-protein docking. Nature Protocols, 12(2), 255-278.), the molecular docking protocol was performed, from which the five best scoring interaction models were selected. Based on these five models, a molecular dynamics study was performed using AMBER 18 software with ff19SB 18 force field and calculation of binding free energies (ΔG) for all generated complexes was performed using MMPBSA 19 protocol (Genheden, S., & Ryde, U. (2015). The MM/PBSA and MM/GBSA methods to estimate ligand-binding affinities. Expert Opinion on Drug Discovery, 10(5), 449-461).

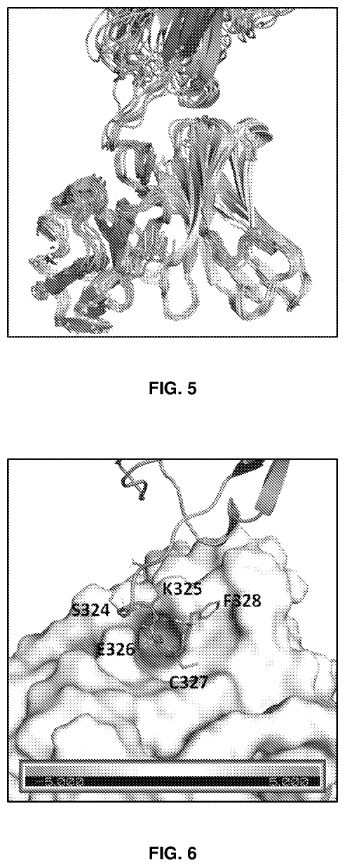

There are three interaction models that presented favorable ΔG for the binding of both proteins and that, in addition, share an electrostatic interaction center conformed by residues: R100, G101, L102, V103 and R104 of scFv with residue E326 of the sST2 protein. This type of electrostatic interaction is clearly visualized in the surface analysis shown in FIG. 5, where the center of attraction for charges around which other relevant interactions between the two proteins are generated is observed. In this model, the different positions taken by the sST2 ligand (green, upper sector) and its epitope (red, middle sector just below green) with respect to the CDRs (cyan and yellow, middle sectors) of scFv-αsST2 (blue, lower sector) are observed. As expected for a complex with high binding energy, the spatial positions do not vary significantly in the time course of the dynamics, strongly suggesting that the epitope is stably anchored in the paratope conformed by the CDRs of the scFv-αsST2. The model obtained for complex 5 is the one that showed the least movement among the side chains throughout the course of the molecular dynamics (FIG. 6), showing a positively charged electrostatic surface on the scFv-αsST2 located on the CDR3 of the heavy chain (H3) that can strongly interact with the negatively charged E326 residue found on the sST2 epitope. Around this anchor point, using a radius of 5 Å, possible interactions of relevance for the analyzed complexes were evaluated, which agrees with a calculated ΔG value of 20.7±5.2 kcal/mol, which was the lowest for all the evaluated complexes. Finally, using an alanine scanning protocol it was possible to verify that upon mutating the E326 residue for alanine, the interaction free energy dropped significantly, indicating that the interactive interaction center observed in FIG. 5 would be of great relevance in the binding mechanism between the two proteins.

### Example 3: Evaluation of the Detection Capacity and Specificity of the scFv by the sST2 Molecule

In parallel, to evaluate the detection capacity and specificity of scFv by the sST2 molecule, in vitro immunoprecipitation and flow cytometry assays were performed using HMC-1 cells, which produce the two isoforms of the ST2 protein, sST2 and ST2L.

To detect the native sST2 protein, immunoprecipitation was performed using the supernatant of phorbol 12-myristate 13-acetate (PMA)-stimulated HMC-1 cells (120 mL), concentrated 100-fold by ultrafiltration. Then, the concentrated supernatant was incubated with scFv 96-1, the anti-HA antibody, and this complex was immunoprecipitated using A/G protein. Proteins were resolved on a polyacrylamide gel and then transferred to a nitrocellulose membrane, which was blocked and incubated with the aST2 antibody (R&D Systems, Inc., 614 Mckinley Place NE Minneapolis, MN 55413, #AF523), and subsequently with anti-goat HRP secondary antibody.

Briefly, cells were seeded in culture and stimulated with PMA (10 ng/ml) for 24 hours, which is a pro-inflammatory stimulus that induces expression and secretion of the sST2 protein. Subsequently, the supernatant was harvested and incubated with scFv-αsST2, an anti-HA antibody having a hemagglutinin (HA) tag, and a A/G protein (Invitrogen) was used to immunoprecipitate this complex. The immunoprecipitation was then loaded onto a polyacrylamide gel, and incubated with a commercial anti-ST2 antibody (R&D Systems, Inc., 614 Mckinley Place NE Minneapolis, MN 55413, #AF523) to perform an Immunoblot.

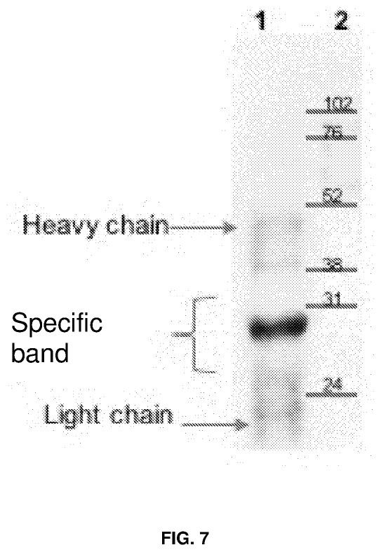

From these experiments, two bands of approximately 50 kDa and approximately 20 kDa, respectively, were observed, which correspond to the heavy and light chain, respectively, of the anti-HA antibody. Additionally, a band of approximately 30 kDa corresponding to the production of native sST2 by HMC-1 cells was observed (FIG. 7). This strongly suggests that scFv can detect the native sST2 molecule.

On the other hand, a cytometry assay was performed to evaluate the specificity of the scFv-αsST2 molecule, where scFv-αsST2 was labeled with a fluorophore in permeabilized and non-permeabilized HMC-1 cells, and in addition a commercial anti-sST2 polyclonal antibody (R&D) that recognizes both isoforms (ST2L and the sST2) was used.

HMC-1 cells were stimulated with PMA (10 ng/ml for 16 hours) to induce sST2, and Brefeldin A was used to inhibit protein secretion for 4 hours. Subsequently, cells were fixed with paraformaldehyde (2%) and permeabilized with permeabilization buffer (BD Biosciences). The scFv-αsST2 was labeled with a fluorophore to be detected by flow cytometry (DyLight488 conjugation Kit Abcam) and cells were incubated for 15 minutes with 0 and 20 μg of scFv-αsST2. The APC-conjugated commercial anti-sST2 polyclonal antibody (Ac-αhST2 R&D), which detects both isoforms of ST2, was used. In addition, an isotype control (Ctol) was used, and the cells were subsequently read using flow cytometry equipment.

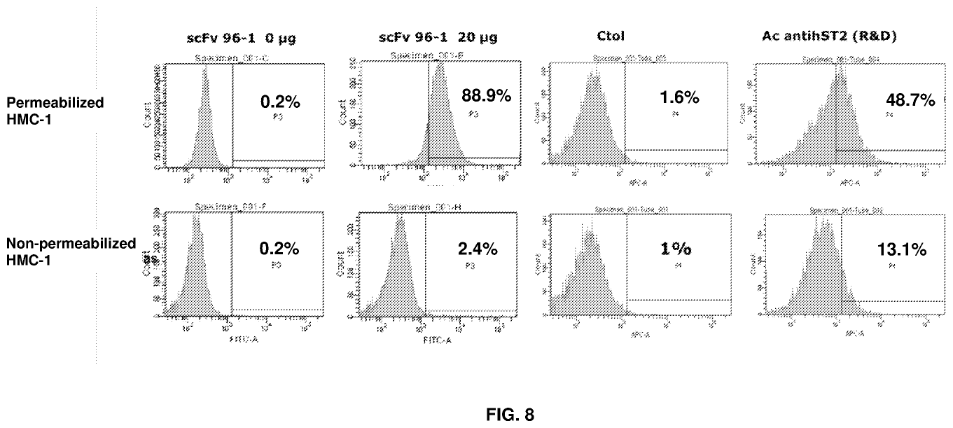

This assay showed that in unpermeabilized cells (which determines labeling only of the ST2L membrane receptor) the commercial antibody recognized the ST2L molecule (13.2%), whereas the scFv-αsST2 had minimal labeling in the cell population (2.4%) (FIG. 8). The scFv-αsST2 significantly labeled the interior of cells when they were permeabilized (88.9%). This result suggests that scFv-αsST2, unlike the commercial anti-sST2 antibody, has a higher specificity for the sST2 variant than for the ST2L receptor.

Additionally, the capacity of scFv-αsST2 to recognize a recombinant sST2 protein (produced in E. coli BL21-(DE3)) was assessed by an ELISA assay where the dissociation constant (K_{D}) between scFv and sST2 was calculated.

For the ELISA assay, a 96-well plate (ThermoFisher, Immunoplate, MaxiSorp) was sensitized with purified recombinant sST2 protein (40, 20, 10, 5, 2.5, 1.25, 0 ug/ml), in triplicate with a volume of 100 μL per well, and incubated overnight at 4 °C. The plate was washed 3 times with buffer solution (PBS tween 0.05%) and blocked with 1% PBS/BSA (200 μL) 1 hour at 37 °C in humid chamber. The plate was then washed 5 times with PBS tween 0.05% (200 μL).

The scFv-αsST2 was purified under denaturing conditions and re-naturalized in 50 mM Tris-HCl buffer+500 mM arginine. This scFv was concentrated by ultrafiltration and the protein concentration was assessed by Bradford's method.

The scFv-αsST2 96-1 was added to the plate (diluted in PBS-tween 0.05% BSA 1%) at the different concentrations (40, 20, 10, 5, 2.5, 1.25, 0 μg/ml) at 100 μL per well 1 hour at 37 °C. Five washes were performed with 0.05% PBS-tween buffer.

Anti-HA antibody (ThermoFisher) 1:7,500 in PBS-tween BSA 1%, was incubated for 1 hour at 37 °C, whereupon five washes were performed. HRP rabbit-mouse secondary antibody (ThermoFisher) 1:5000 in PBS-tween BSA was added and incubated for 1 hour at 37 °C, and then washed 5 times. 100 μL of 3,3′,5,5′-tetramethylbenzidine (TMB) substrate solution, was added to each well.

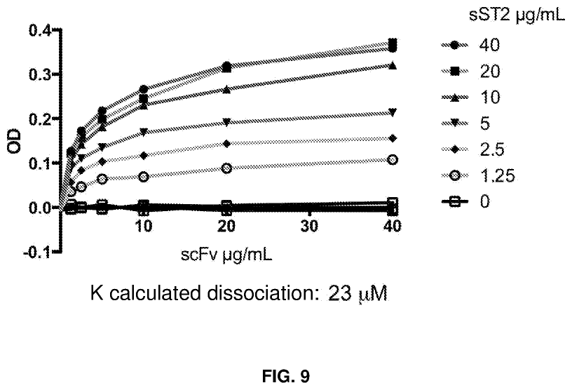

After 20 min, 50 μL of the 2N(H₂SO₄) stop solution was added and the optical density (OD) was read at a wavelength of 450 nm (Synergy 2). The K_{D} determined by the method of Beatty and Beatty (Beatty, J. D., Beatty, B. G., & Vlahos, W. G. (1987). Measurement of monoclonal antibody affinity by non-competitive enzyme immunoassay. Journal of Immunological Methods, 100(1-2), 173-179) was 23 μM as seen in FIG. 9, a value that is within the expected range for a scFv obtained from a phage library. This result also demonstrates that the scFv can recognize the recombinant sST2 protein.

Additionally, the K_{D} between ScFv and the SKECF differentiating peptide of sST2 was evaluated using the surface plasmon resonance technique, which is the U.S. Food and Drug Administration (FDA)-approved technique for evaluating K_{D} between molecules.

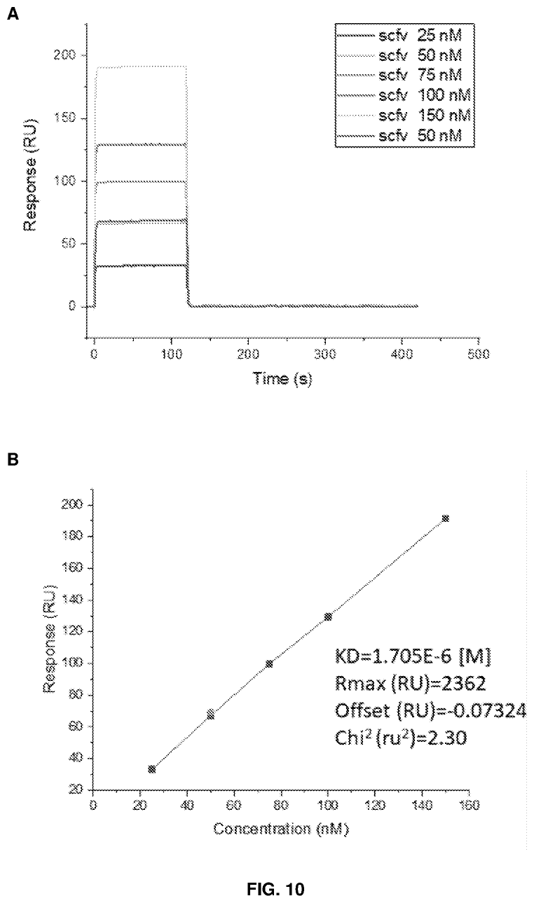

For this purpose, the SKECF peptide was immobilized on the surface of the SA sensor chip, and then confronted with increasing concentrations of scFv through a microfluid that was passed through the sensor. In FIG. 10A, the sensogram for each scFv concentration that was tested is observed, where each curve represents the stages of association and dissociation between the molecules on a graph representing the measured Resonance Units (RU) as a function of time in seconds (s). Using the data obtained from the sensogram, a RU vs concentration curve was constructed, and from the linear regression analysis the K_{D} value was obtained, which in this case was 1.705 μM which is in an expected range for a scFv and can be seen in FIG. 10B. This result is consistent with a previous observation made by ELISA, where a K_{D} value of 23 μM was obtained.

### Example 4. Design and Production of a Complete Antibody Directed Against sST2

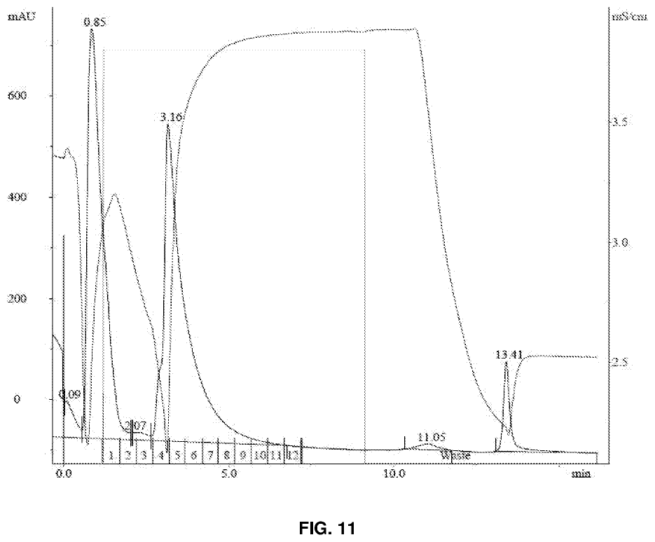

Subsequent to these assays, the complete antibody directed against sST2 (AcHu-αsST2) was designed and produced from the amino acid sequences of scFv-αsST2. The AcHu-αsST2 was produced in cells transfected with two expression vectors, the first containing the variable and constant heavy chain sequence cloned into the pCI_MSC_LGL vector containing the GFP molecule (Remington, S. J. (2011). Green fluorescent protein: A perspective. Protein Science, 20(9), 1509-1519.). The variable light chain followed by the Kappa sequence was cloned into a commercial Pcl-Neo vector (Promega, Madison WI, USA, Genbank/EMBL pCl-neo mammalian expression vector accession code U47120). Both vectors were co-transfected into CHO-K1 cells for the production of AcHu-αsST2. The antibody thus obtained was purified from the supernatant of CHO-K1 cells by fast protein liquid chromatography (FPLC) using protein G columns (Hitrap GE), with the ÄKTAPure kit. The column was equilibrated with phosphate buffer pH 7.4, and the antibody was eluted with Glycine-HCl solution (pH 2.7). The team collected 12 elutions with a volume of 500 μL each. The chromatogram in FIG. 11 shows the elution of the antibody from the protein G matrix.

The peak reading of 3.16 corresponds to the highest elution of the AcHu-αsST2 antibody, which was obtained between collection tubes 5-6 as seen in said Figure, where the line having peaks marked with numbers representing the minutes corresponds to the UV meter; the line having a sharp drop after 11 minutes is the pH indicator; and the remaining line with a binary signal (with a value 0 until minute 1, and 0 again past minute 9) is the elution segment. Subsequently, the eluted antibody was quantified by ELISA technique, where an AcHu-αsST2 concentration of 29.7 μg/mL and a total protein amount of 89.3 μg was obtained.

### Example 5. Evaluation of the Capacity of AcHu-αsST2 to Reverse Inflammatory Parameters in Ex Vivo Assays

With the purified antibody, the capacity of AcHu-αsST2 to reverse inflammatory parameters was evaluated through ex vivo assays using colonoscopy biopsies from patients suffering from UC and control individuals without inflammatory bowel disease. This assay allows to evaluate the effect of drugs on cytokine production by different cell types present in the biopsy (epithelial cells, endothelial cells, lymphocytes, macrophages) so it could partly reflect the effect that the therapy would have on patients in vivo.

Seven control individuals and three UC patients were enrolled. The latter had a moderate to severe degree of inflammation at the intestinal mucosal level according to the Mayo endoscopic index (Colonoscopic evaluation in ulcerative colitis. Elizabeth R. Paine. Gastroenterology Report, Volume 2, Issue 3, August 2014, Pages 161-168, https://doi.org/10.1093/gastro/gou028). Four biopsies were taken during the colonoscopic procedure performed by the treating physician to control pathology (UC patients) or by the cancer prevention program (control individuals). The protocols for obtaining samples were approved by the Ethics Committee on Human Subjects of the Clinica Las Condes and the Faculty of Medicine of the Universidad de Chile, and all patients signed informed consent.

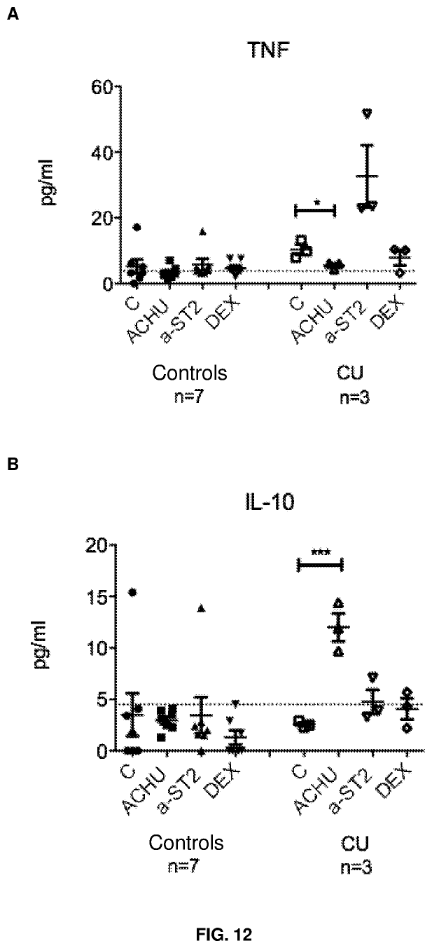

Biopsies or explants were stimulated for 24 hours with the AcHu-αsST2 (5 μg/mL), the commercial Ac-ST2 (5 μg/mL) (R&D), a synthetic glucocorticoid as an anti-inflammatory control (Dex, 100 nM) (Díaz-Jiménez D, Núñez L, De la Fuente M, Dubois K, Torres A, García-González P, Chnaiderman J, Sepúlveda H, Montecino M, Vossenkaemper A, MacDonald T T, Simian D, González M J, Quera R, Hermoso M A. A functional IL1RL1 variant regulates corticosteroid-induced sST2 expression in ulcerative colitis. Scientific Rep 2017; 7(1):10180. doi: 10.1038/s41598-017-10465-0) and a biopsy was used as an unstimulated control. Pro- and anti-inflammatory cytokines (TNF and IL-10, respectively) were assessed from the supernatants by Cytometric Beads Array (CBA) technique (Landskron G, Dubois-Camacho K, Orellana-Serradell O, De la Fuente M, Parada-Venegas D, Bitrán M, Diaz-Jimenez D, Tang S, Cidlowski J A, Li X, Molina H, Gonzalez C M, Simian D, Lubascher J, Pola V, Montecino M, Blokzijl T, Faber K N, González M J, Quera R, Hermoso M A. Regulation of the Intestinal Extra-Adrenal Steroidogenic Pathway Component LRH-1 by Glucocorticoids in Ulcerative Colitis. Cells. 2022 Jun. 12; 11(12):1905. doi:10.3390/cells11121905.). TNF and IL-10 levels were subsequently quantified using the CBA technique. (One-way ANOVA, *p<0.5; **p<0.001). The results show that AcHu-αsST2 decreases TNF levels as can be seen in FIG. 12A and increases those of IL-10, significantly as can be seen in FIG. 12B. This effect was not observed with the commercial anti-ST2 antibody, possibly because the latter can bind to the ST2L receptor preventing the reparative action of IL-33. This result was not observed in samples from control individuals, possibly because there was no inflammatory component on which AcHu-αsST2 exerted its effect. This result reflects the capacity of AcHu-αsST2 to decrease a proinflammatory cytokine and promote an inflammation-regulating cytokine, an effect that is desired in therapies used in patients with UC and other pathologies. This result also suggests that AcHu-αsST2 would specifically bind to sST2, discriminating the isoforms of the protein, allowing the binding of IL-33 to its ST2L receptor, promoting its anti-inflammatory reparative effect.

These results in the ex vivo model used are very promising and make this antibody a future therapeutic candidate in ulcerative colitis and potentially for other diseases with an inflammatory component.

### Example 6. Study of Site-Directed Mutation Variants for Increased Affinity Between scFv-αsST2 and the sST2 Protein

The mini antibody or single-chain variable fragment (scFv) that specifically binds to the SKECF peptide present in the soluble ST2 (sST2) protein or soluble IL-33 receptor, referred to in this document as scFv-αsST2, was used as a basis for the design of new site-directed mutation variants that increase the affinity for the sST2 protein. The advantage of using a mini scFv antibody, is the possibility to obtain large amounts (on the order of mg) of protein in a short time and at low cost in prokaryotic systems. For the proposal of scFv-αsST2 mutation variants, a rational design was performed using molecular modeling though the use of different software (in silico).

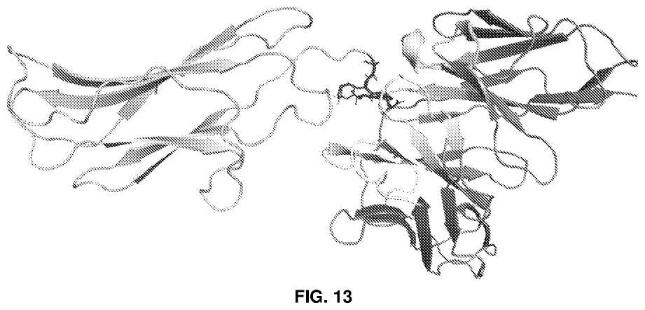

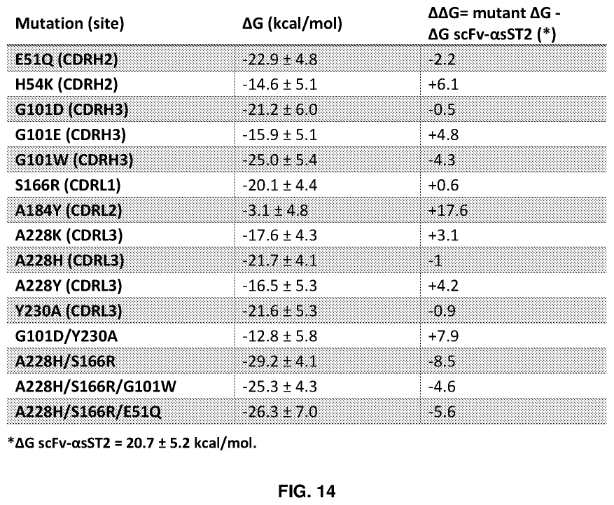

The in silico study started with the molecular modeling of sST2 and scFv-αsST2 proteins. For the case of sST2, the homology modeling method was used (Song Y, Dimaio F, Wang R Y R, Kim D, Miles C, Brunette T, et al. High-resolution comparative modeling with RosettaCM. Structure [Internet]. 2013; 21(10):1735-42. Retrieved from: http://dx.doi.org/10.1016/j.str.2013.08.005), using as a template (pattern) the crystal structure of the extracellular domain of the ST2L protein (available at: https://www.rcsb.org/structure/4KC3), having a sequence homology close to 100% to sST2 (Liu X, Hammel M, He Y, Tainer J A, Jeng U S, Zhang L, et al. Structural insights into the interaction of IL-33 with its receptors. Proc Natl Acad Sci USA. 2013; 110(37):14918-23). On the other hand, the structural model of scFv-αsST2 was obtained using homology modeling tools complemented by ab initio (from source) modeling using the theoretical functionality of the protein of interest from a database (Roy A, Kucukural A, Zhang Y. I-TASSER: A unified platform for automated protein structure and function prediction. Nat Protoc. 2010; 5(4):725-38). The protein-protein interaction analysis was initiated with a molecular docking modeling study on the ClusPro 2.0 platform (Brenke R, Hall D R, Chuang G-Y, Comeau S R, Bohnuud T, Beglov D, Schueler-Furman O, Vajda S, Kozakov D. Application of asymmetric statistical potentials to antibody-protein docking. Bioinformatics. 2012 October; 28(20):2608-2614), using the antigen (sST2)/antibody (scFv-αsST2) docking function. Evaluation of the approach poses between the sST2 epitope (SKECF peptide) and the complementarity determining regions (CDRs) forming the paratope was proposed. In FIG. 13, the protein-protein interaction model is observed; in green (left) the structural model of the sST2 protein with its epitope is visualized: SKECF highlighted in red (center) toward the carboxyl-terminal end; in blue (right) the framework structure of the scFv-αsST2 is visualized; and highlighted in yellow (lower right center) and light blue (upper right center) the paratope composed of the CDRs of the chains: heavy and light. After obtaining the interaction models, a molecular dynamics study was performed using AMBER 18 software (University of California, San Francisco Dept. of Pharmaceutical Chemistry 600 16th St. 5th floor, Room GH-518A Box 2280, Mission Bay Campus San Francisco, CA 94158-2517), which allowed the calculation of binding free energy (ΔG) for the complexes using the MM-PBSA protocol (Roy A, Kucukural A, Zhang Y. I-TASSER: A unified platform for automated protein structure and function prediction. Nat Protoc. 2010; 5(4):725-38; Kozakov D, Hall D R, Xia B, Porter K A, Padhorny D, Yueh C, et al. The ClusPro web server for protein-protein docking. Nat Protoc. 2017; 12(2):255-78; D. A. Case, et al. AMBER 18. San Francisco: University of California; 2018.). Among all the interaction complexes evaluated, the complex that yielded the most favorable ΔG value was selected. The theoretical binding energy (ΔG) value calculated for this interaction complex (between scFv-αsST2 and sST2), was −20.7±5.2 kcal/mol. Subsequently, using molecular dynamics information, site-directed mutations were proposed for the purpose of favoring binding energy (the more negative the ΔG, the more favorable the binding). Once the amino acid changes were introduced, the binding energy calculation was performed for each of the proposed site-directed mutation variants as can be seen in FIG. 14.

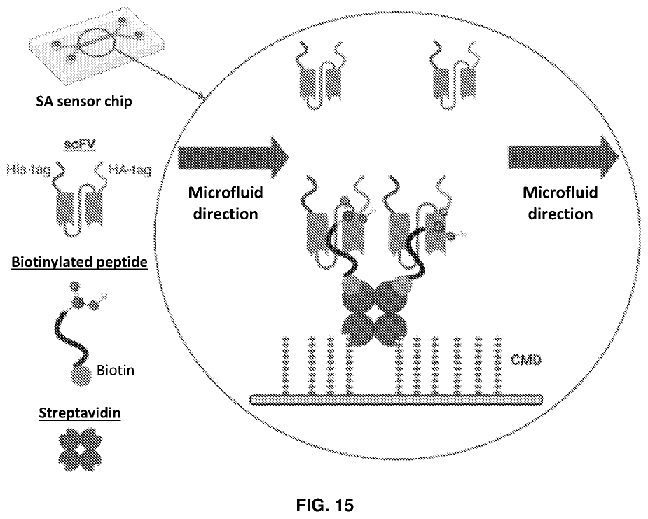

For experimental evaluation of the affinity between the original mini scFv-αsST2 antibody or its mutation variants (E51Q, G101W, A184Y and A228H/S166R) and the sST2 protein, a Biacore kit (Biacore AB Corporation, Uppsala, Sweden) was used. The principle is based on a physical property known as surface plasmon resonance (SPR). A sensor chip with a surface functionalized with streptavidin molecules was used for the measurements. First, a biotinylated peptide (SKECF) representative of the sST2 protein epitope was attached, and subsequently, the response to different increasing concentrations of solutions of the scFv-αsST2 and its mutation variants, which pass through the sensor in a vehicle (microfluid), was assayed giving as response a signal represented as resonance units (FIG. 15). Using the maximum resonance units (RUₘₐₓ) for each different scFv concentration as a function of time measured in seconds, concentration versus RUₘₐₓ curves were constructed with which the K_{DS} for the scFv-αsST2 and its mutation variants could be calculated, thus obtaining the affinities for each mutant as can be seen in FIG. 16.

1. An isolated antibody or fragment thereof that binds to a peptide having an amino acid sequence SKECF (SEQ ID NO: 1), wherein said isolated antibody or fragment comprises:
a) a heavy-chain variable domain comprising:
i. a CDR:H1 having an amino acid sequence as set forth in SEQ ID NO: 2;
ii. a CDR:H2 having an amino acid sequence as set forth in SEQ ID NO: 3; and
iii. a CDR:H3 having an amino acid sequence as set forth in SEQ ID NO: 4;
and
b) a light-chain variable domain comprising:
i. a CDR:L1 having an amino acid sequence as set forth in SEQ ID NO: 5;
ii. a CDR:L2 having an amino acid sequence as set forth in SEQ ID NO: 6; and
iii. a CDR:L3 having an amino acid sequence as set forth in SEQ ID NO: 7.

2. The isolated antibody or fragment thereof according to claim 1, wherein said peptide SKECF is comprised within a protein or polypeptide.

3. The isolated antibody or fragment thereof according to claim 2, wherein said protein is the Suppresion of tumorigenicity-2 (ST2) protein, or any of its variants or isoforms.

4. The isolated antibody or fragment thereof according to claim 3, wherein said protein is the isoform sST2 of the ST2 protein.

5. The isolated antibody or fragment thereof according to claim 4, wherein said sST2 protein is a mammalian sST2 protein.

6. The isolated antibody or fragment thereof according to claim 5, wherein said mammalian sST2 protein is selected from the group of human sST2 protein, bovine sST2 protein, porcine sST2 protein, ovine sST2 protein, caprine sST2 protein or rodent sST2 protein.

7. The isolated antibody or fragment thereof according to claim 6, wherein said mammalian sST2 protein is a human sST2 protein having an amino acid sequence as set forth in SEQ ID NO: 2.

8. The isolated antibody or a fragment thereof according to claim 1, wherein said CDR:H2 having an amino acid sequence as set forth in SEQ ID NO: 4 further comprises a substitution of glutamic acid (E) for glutamine (Q) at position 1.

9. The isolated antibody or fragment thereof according to claim 1, wherein said CDR:H3 having an amino acid sequence as set forth in SEQ ID NO: 5 further comprises a substitution of glycine (G) for tryptophan (W) at position 5.

10. The isolated antibody or a fragment thereof according to claim 1, wherein said heavy-chain variable domain comprises the sequence of SEQ ID NO: 8.

11. The isolated antibody or a fragment thereof according to claim 10, wherein said heavy-chain variable domain further comprises at least one substitution in the amino acid sequence of SEQ ID NO: 9 selected from the group consisting of: glutamic acid (E) for glutamine (Q) at position 51, and glycine (G) for tryptophan (W) at position 101.

12. The isolated antibody or a fragment thereof according to claim 1, wherein said CDR:L1 having an amino acid sequence as set forth in SEQ ID NO: 6 further comprises a substitution of serine(S) for arginine (R) at position 10.

13. The isolated antibody or a fragment thereof according to claim 1, wherein said CDR:L2 having an amino acid sequence as set forth in SEQ ID NO: 7 further comprises a substitution of alanine (A) for tyrosine (Y) at position 1.

14. The isolated antibody or a fragment thereof according to claim 1, wherein said CDR:L3 having an amino acid sequence as set forth in SEQ ID NO: 8 further comprises a substitution of alanine (A) for histidine (H) at position 6.

15. The isolated antibody or a fragment thereof according to claim 1, wherein said isolated antibody or fragment further comprises:
a substitution of serine(S) for arginine (R) at position 10 in the CDR:L1, having an amino acid sequence as set forth in SEQ ID NO: 6; and,
a substitution of alanine (A) for histidine (H) at position 6 in the CDR:L3, having an amino acid sequence as set forth in SEQ ID NO: 8.

16. The isolated antibody or a fragment thereof according to claim 1, wherein said light-chain variable domain comprises the sequence of SEQ ID NO: 9.

17. The isolated antibody or a fragment thereof according to claim 16, wherein said light-chain variable domain further comprises at least one substitution in the amino acid sequence of SEQ ID NO: 10, selected from the group consisting of:
serine(S) for arginine (R) at position 33;
alanine (A) for tyrosine (Y) at position 51; and,
alanine (A) for histidine (H) at position 95.

18. The isolated antibody or a fragment thereof according to any one of the preceding claims, wherein said isolated antibody or fragment further comprises an amino acid sequence selected from the group consisting of: SEQ ID NO: 10, SEQ ID NO: 11, SEQ ID NO: 12, SEQ ID NO: 13, or SEQ ID NO: 14.

19. The isolated antibody or a fragment thereof according to any one of the preceding claims, wherein said isolated antibody is a human IgG antibody.

20. The isolated antibody or a fragment thereof according to claim 18, wherein said isolated antibody is a single-chain antibody fragment (scFv) of the heavy and light variable regions of a human IgG.

21. An isolated nucleic acid, wherein said isolated nucleic acid encodes for an isolated antibody or a fragment thereof of any one of claims 1 to 20.

22. The isolated nucleic acid of claim 21, wherein said isolated nucleic acid comprises a nucleotide sequence encoding for an isolated antibody or a fragment thereof, comprising:
a) a heavy-chain variable domain comprising:
i. a CDR:H1 having an amino acid sequence as set forth in SEQ ID NO: 15;
ii. a CDR:H2 having an amino acid sequence as set forth in SEQ ID NO: 16; and
iii. a CDR:H3 having an amino acid sequence as set forth in SEQ ID NO: 17;
and
b) a light-chain variable domain comprising:
i. a CDR:L1 having an amino acid sequence as set forth in SEQ ID NO: 18;
ii. a CDR:L2 having an amino acid sequence as set forth in SEQ ID NO: 19; and
iii. a CDR:L3 having an amino acid sequence as set forth in SEQ ID NO: 20.

23. The isolated nucleic acid of claim 23, wherein said isolated nucleic acid further comprises:
a) a sequence encoding the heavy-chain variable domain comprising:
i. a sequence encoding the CDR:H1, having a nucleotide sequence as set forth in SEQ ID NO: 21;
ii. a sequence encoding the CDR:H2, having a nucleotide sequence as set forth in SEQ ID NO: 22; and
iii. a sequence encoding the CDR:H3, having a nucleotide sequence as set forth in SEQ ID NO: 23;
and
b) a sequence encoding the light-chain variable domain comprising:
i. a sequence encoding the CDR:L1, having a nucleotide sequence as set forth in SEQ ID NO: 24;
ii. a sequence encoding the CDR:L2, having a nucleotide sequence as set forth in SEQ ID NO: 25;
iii. a sequence encoding the CDR:L3, having a nucleotide sequence as set forth in SEQ ID NO: 26.

24. The isolated nucleic acid of claim 22, wherein said isolated nucleic acid comprises a nucleotide sequence encoding for the heavy chain variable domain, having an amino acid sequence as set forth in SEQ ID NO: 9, SEQ ID NO: 33 or SEQ ID NO: 34; and a nucleotide sequence encoding for the light chain variable domain, having an amino acid sequence as set forth in SEQ ID NO: 10, SEQ ID NO: 35 or SEQ ID NO: 36.

25. The isolated nucleic acid of claim 24, wherein said isolated nucleic acid further comprises a nucleotide sequence coding for the heavy chain variable domain, selected from the group consisting of: SEQ ID NO: 27, SEQ ID NO: 28, AND SEQ ID NO: 29; and a nucleotide sequence coding for the light chain variable domain, selected from the group consisting of: SEQ ID NO: 30, SEQ ID NO: 31, AND SEQ ID NO: 32.

26. The isolated nucleic acid of claim 22, wherein said isolated nucleic acid comprises a nucleotide sequence encoding for an isolated antibody, having an amino acid sequence as set forth in SEQ ID NO: 11, SEQ ID NO: 12, SEQ ID NO: 13, SEQ ID NO: 14, or SEQ ID NO: 15.

27. The isolated nucleic acid of claim 26, wherein said isolated nucleic acid further comprises a nucleotide sequence selected from the group consisting of: SEQ ID NO: 33, SEQ ID NO: 34, SEQ ID NO: 35, SEQ ID NO: 36, OR SEQ ID NO: 37.

28. The isolated nucleic acid of any one of claims 21 to 27, wherein said isolated nucleic acid comprises DNA, RNA or any of its derivatives.

29. A pharmaceutical composition, comprising a therapeutically effective quantity of an antibody or fragment thereof of any one of claims 1 to 20; and a pharmaceutically acceptable excipient.
